(19) United States
(12) Patent Application Publication
(10) Pub. No.: US 2023/0135175 A1
(43) Pub. Date: May 4, 2023

(54) Feedback For Improving Diabetes Management

(71) Applicant: Dexcom, Inc., San Diego, CA (US)

(72) Inventors: Lauren H. Jepson, San Diego, CA (US); Margaret A. Crawford, San Diego, CA (US); Mark Derdzinski, San Diego, CA (US); Robert J. Dowd, San Diego, CA (US); Giada Acciaroli, San Diego, CA (US); Sarah Kate Pickus, San Diego, CA (US); Apurv U. Kamath, San Diego, CA (US)

(73) Assignee: Dexcom, Inc., San Diego, CA (US)

(21) Appl. No.: 17/974,185

(22) Filed: Oct. 26, 2022

Related U.S. Application Data
(60) Provisional application No. 63/263,188, filed on Oct. 28, 2021.
(60) Provisional application No. 63/292,929, filed on Dec. 22, 2021.

(51) Int. Cl. A61B 5/00; G16H 70/20; A61B 5/145; A61B 5/11

(52) U.S. Cl. CPC A61B 5/486; G16H 70/20; A61B 5/14532; A61B 5/4866; A61B 5/1118; A61B 5/742

(57) ABSTRACT

Glucose level measurements or other data regarding a user are obtained over time, such as from a wearable glucose monitoring device being worn by the user. These glucose level measurements or other data are analyzed based on various rules to determine time periods during a day of, for example, good diabetes management by the user and provide feedback indicating such to the user. Good diabetes management is identified in various manners, such as by identifying improvements in glucose measurements for a given time period over one or more previous days, identifying a time period of the day during which glucose measurements were the best, identifying sustained positive patterns (e.g., good diabetes management for a same time period in each of multiple days), and so forth.

### RELATED APPLICATIONS

This application claims the benefit of U.S. Provisional Patent Application No. 63/292,929, filed Dec. 22, 2021, and titled “Feedback For Improving Diabetes Management,” the entire disclosure of which is hereby incorporated by reference, and also claims the benefit of U.S. Provisional Patent Application No. 63/263,188, filed Oct. 28, 2021, and titled “Ranking Feedback For Improving Diabetes Management,” the entire disclosure of which is hereby incorporated by reference.

### BACKGROUND

Diabetes is a metabolic condition affecting hundreds of millions of people and is one of the leading causes of death worldwide. For people living with Type I diabetes, access to treatment is critical to their survival and it can reduce adverse outcomes among people with Type II diabetes. With proper treatment, serious damage to the heart, blood vessels, eyes, kidneys, and nerves due to diabetes can be avoided. Regardless of the type of diabetes (e.g., Type I or Type II), managing diabetes successfully involves monitoring and oftentimes adjusting food and activity to control a person's blood glucose, such as to reduce severe fluctuations in and/or generally lower the person's glucose.

However, many conventional glucose monitoring applications employ user interfaces that display raw glucose information in a manner that is difficult for users to interpret, particularly users who have just recently started monitoring their glucose. Consequently, users may be unable to draw insights from the data and thus are unable to alter their behavior in a meaningful way in order to improve their glucose. Over time, these users often become overwhelmed and frustrated by the manner in which information is presented by these conventional glucose monitoring applications and thus discontinue use of these applications before improvements in their glucose and overall health can be realized. Moreover, as users increasingly utilize mobile devices (e.g., smart watches and smart phones) to access glucose monitoring information, the failure by conventional systems to provide meaningful glucose information in a manner that users can understand is further exacerbated by the constraints imposed by the small screens of these mobile devices.

### SUMMARY

To overcome these problems, techniques for diabetes management feedback for improving diabetes management are discussed. In one or more implementations, in a continuous glucose level monitoring system, first glucose measurements are obtained from a glucose sensor of the continuous glucose level monitoring system. The first glucose measurements are measured for a user for a first time period of multiple time periods of a current day, the glucose sensor being inserted at an insertion site of the user. One or more features are generated from the first glucose measurements for the first time period of the current day. The one or more features are analyzed to determine at least one feature that satisfies one or more rules for the first time period of the current day. Which of multiple diabetes management feedbacks in a library of diabetes management feedbacks that corresponds to the satisfied one or more rules is identified. A user interface including the identified diabetes management feedback as well as an indication of the first time period is generated, the identified diabetes management feedback is caused to be displayed.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example of an implementation that is operable to implement diabetes management feedback for improving diabetes management as described herein.

FIG. 2 depicts an example of an implementation of a wearable glucose monitoring device in greater detail.

FIG. 3 is an illustration of an example architecture of a diabetes management feedback generation system.

FIG. 4 illustrates an example of providing feedback indicating improvement in at least one feature for a time period.

FIG. 5 illustrates an example of providing feedback indicating a best time period during a day.

FIG. 6 illustrates an example of providing feedback indicating a sustained positive pattern.

FIG. 7 depicts a procedure in an example of implementing diabetes management feedback for improving diabetes management.

FIG. 8 depicts a procedure in another example of implementing diabetes management feedback for improving diabetes management.

FIG. 9 illustrates an example of a system generally that includes an example of a computing device that is representative of one or more computing systems and/or devices that may implement the various techniques described herein.

### DETAILED DESCRIPTION

Overview

Techniques for feedback for improving diabetes management are discussed herein. Broadly, blood glucose level measurements of a user are obtained over time. Glucose level measurements are typically obtained by a wearable glucose monitoring device being worn by the user. These glucose level measurements can be produced substantially continuously, such that the device may be configured to produce the glucose level measurements at regular or irregular intervals of time (e.g., approximately every hour, approximately every 30 minutes, approximately every 5 minutes, and so forth), responsive to establishing a communicative coupling with a different device (e.g., when a computing device establishes a wireless connection with a wearable glucose level monitoring device to retrieve one or more of the measurements), and so forth. These glucose level measurements are analyzed based on various rules to determine time periods of good (or optionally bad) diabetes management by the user and feedback indicating such is provided to the user.

In one or more implementations, a data stream of glucose measurements is received. Various other data streams can also optionally be received, such as activity data (e.g., number of steps taken by the user). One or more features for a particular time period are generated and stored, each feature being a value that can be computed from data in a data stream and that indicates whether the user has been engaging in beneficial diabetes management behaviors or lifestyle choices. The features may include metrics that are a representation or summarization of the data in the data stream for a particular time period are generated and stored. These time periods are, for example, different multi-hour blocks of time during a day. E.g., a day may include a first time period from midnight to 6am (corresponding to sleep), a second time period from 6 am to noon (corresponding to after breakfast), a third time period from noon to 6 pm (corresponding to after lunch), and a fourth time period from 6 pm to midnight (corresponding to after dinner). These time periods may be fixed or may be adaptively identified based on features identified in the different data streams (e.g., sleep onset may be detected by an activity monitor and may be used to determine the beginning of the “sleep” time period on that date).

Good diabetes management is identified in various manners, such as by identifying improvements in glucose measurements for a given time period over one or more previous days, identifying a time period of the day during which glucose measurements were the best (e.g., within an optimal range or closest to an optimal value), identifying sustained positive patterns (e.g., good diabetes management for a same time period in each of multiple days), and so forth. Once identified, feedback is generated that notifies the user of these improvements in glucose measurements, best time period of the day, sustained positive patterns, combinations thereof, and so forth. This feedback can be retrospective, e.g., informing the user how he or she did on diabetes management during the previous day or at the end of the current day.

The techniques discussed herein apply analogously to identify bad diabetes management. Poor diabetes management can be identified and feedback (e.g., warnings or negative affects) displayed or otherwise presented to the user to encourage better diabetes management by the user. This feedback can be used to identify problem areas that need to be addressed, actions or choices that should be avoided in the future, and so forth.

The techniques discussed herein generate feedback for the user that notifies the user of good diabetes management choices (or bad diabetes management choices) he or she has made. This feedback is provided for reflection and motivation by the user, oftentimes providing inspirational insights to the user to continue making good diabetes management to improve their health, prolong their life, and so forth. This feedback also educates the user on making good diabetes management choices, for example by allowing the user to mimic his or her changes from one time period that showed improvement (e.g., increasing the amount of vegetables he or she ate) to other time periods.

In some situations, multiple different items of feedback can be provided to the user. In such situations, a prioritization technique is optionally used to prioritize the multiple different items of feedback and select one or two to be provided to the user. This prioritization technique improves the user interface presented to the user by reducing the amount of feedback (the number of items of feedback at once or over time) provided to the user, reducing repetitiveness in the feedback provided to the user, to highlight the largest or most significant glycemic improvements or comparisons, to highlight the glycemic improvements or comparisons most relevant to user at a certain point in time based on contextual information such as diabetes type, comorbidities, medications, patient or clinician preferences, user feedback on previously provided insights, or duration of product use, and so forth.

In the following discussion, an example environment is first described that may employ the techniques described herein. Examples of implementation details and procedures are then described which may be performed in the example environment as well as other environments. Performance of the example procedures is not limited to the example environment and the example environment is not limited to performance of the example procedures.

Example of an Environment

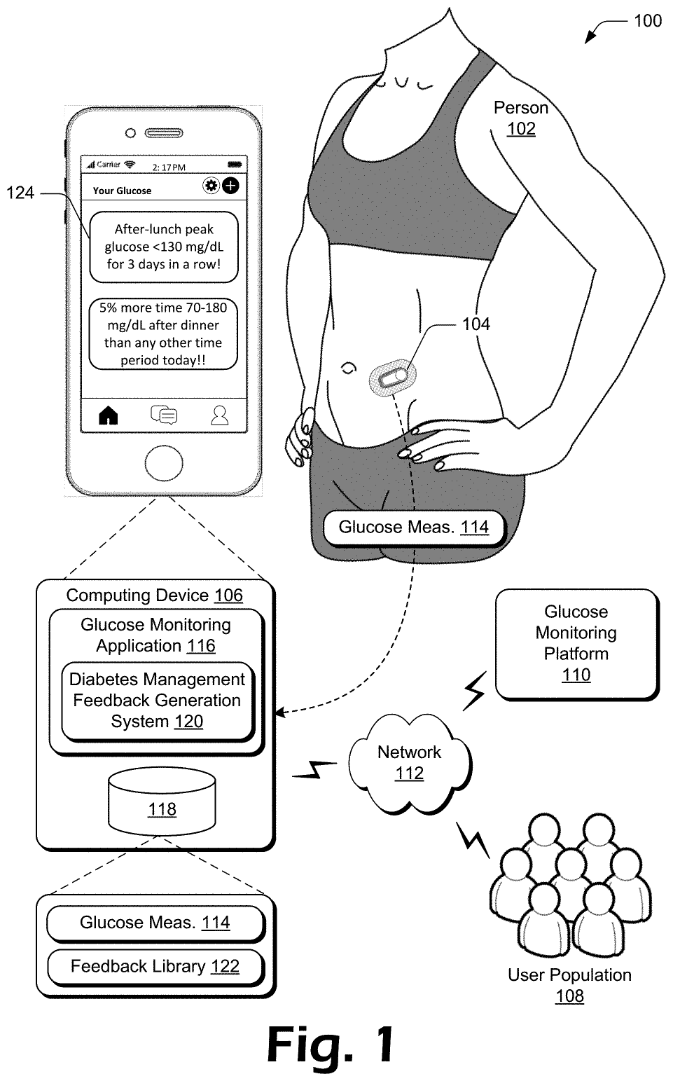

FIG. 1 is an illustration of an environment 100 in an example of an implementation that is operable to implement feedback for improving diabetes management as described herein. The illustrated environment 100 includes a person 102, who is depicted wearing a wearable glucose monitoring device 104. The illustrated environment 100 also includes a computing device 106, other users in a user population 108 that wear glucose monitoring devices 104, and a glucose monitoring platform 110. The wearable glucose monitoring device 104, computing device 106, user population 108, and glucose monitoring platform 110 are communicatively coupled, including via a network 112.

Alternately or additionally, the wearable glucose monitoring device 104 and the computing device 106 may be communicatively coupled in other ways, such as using one or more wireless communication protocols or techniques. By way of example, the wearable glucose monitoring device 104 and the computing device 106 may communicate with one another using one or more of Bluetooth (e.g., Bluetooth Low Energy links), near-field communication (NFC), 5G, and so forth.

In accordance with the described techniques, the wearable glucose monitoring device 104 is configured to provide measurements of person 102's glucose. Although a wearable glucose monitoring device is discussed herein, it is to be appreciated that user interfaces for glucose monitoring may be generated and presented in connection with other devices capable of providing glucose measurements, e.g., non-wearable glucose devices such as blood glucose meters requiring finger sticks, patches, and so forth. In implementations that involve the wearable glucose monitoring device 104, though, it may be configured with a glucose sensor that continuously detects analytes indicative of the person 102's glucose and enables generation of glucose measurements. In the illustrated environment 100 and throughout the detailed description these measurements are represented as glucose measurements 114.

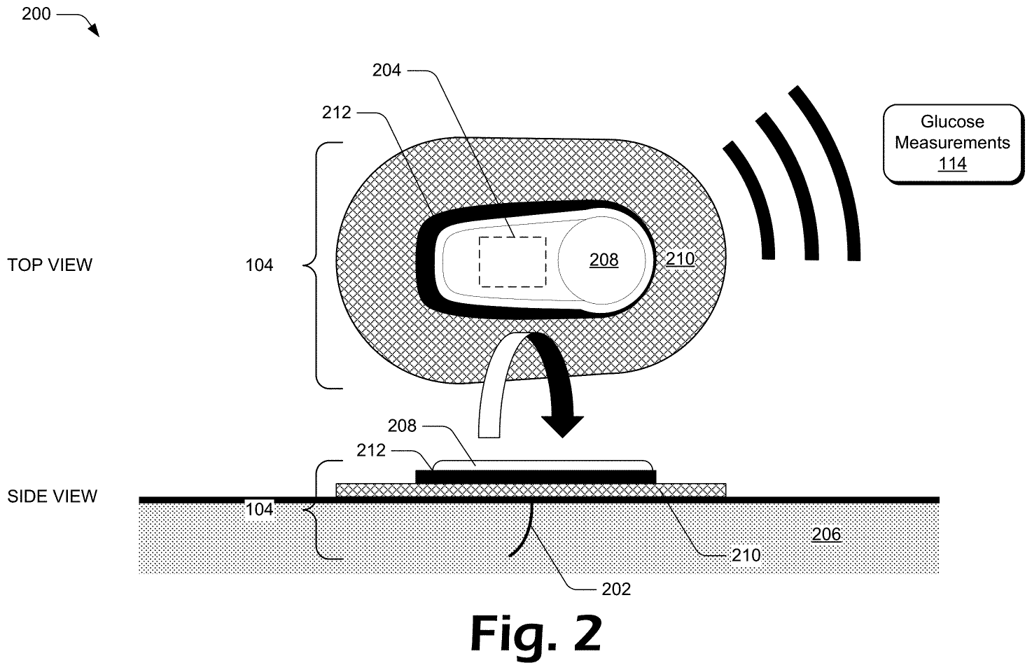

In one or more implementations, the wearable glucose monitoring device 104 is a continuous glucose monitoring (“CGM”) system. As used herein, the term “continuous” used in connection with glucose monitoring may refer to an ability of a device to produce measurements substantially continuously, such that the device may be configured to produce the glucose measurements 114 at regular or irregular intervals of time (e.g., every hour, every 30 minutes, every 5 minutes, and so forth), responsive to establishing a communicative coupling with a different device (e.g., when a computing device establishes a wireless connection with the wearable glucose monitoring device 104 to retrieve one or more of the measurements), and so forth. This functionality along with further aspects of the wearable glucose monitoring device 104's configuration is discussed in more detail in relation to FIG. 2.

Additionally, the wearable glucose monitoring device 104 transmits the glucose measurements 114 to the computing device 106, such as via a wireless connection. The wearable glucose monitoring device 104 may communicate these measurements in real-time, e.g., as they are produced using a glucose sensor. Alternately or in addition, the wearable glucose monitoring device 104 may communicate the glucose measurements 114 to the computing device 106 at set time intervals. For example, the wearable glucose monitoring device 104 may be configured to communicate the glucose measurements 114 to the computing device 106 every five minutes (as they are being produced).

Certainly, an interval at which the glucose measurements 114 are communicated may be different from the examples above without departing from the spirit or scope of the described techniques. The measurements may be communicated by the wearable glucose monitoring device 104 to the computing device 106 according to other bases in accordance with the described techniques, such as based on a request from the computing device 106. Regardless, the computing device 106 may maintain the glucose measurements 114 of the person 102 at least temporarily, e.g., in computer-readable storage media of the computing device 106.

Although illustrated as a mobile device (e.g., a mobile phone), the computing device 106 may be configured in a variety of ways without departing from the spirit or scope of the described techniques. By way of example and not limitation, the computing device 106 may be configured as a different type of device, such as a mobile device (e.g., a wearable device, tablet device, or laptop computer), a stationary device (e.g., a desktop computer), an automotive computer, and so forth. In one or more implementations, the computing device 106 may be configured as a dedicated device associated with the glucose monitoring platform 110, e.g., with functionality to obtain the glucose measurements 114 from the wearable glucose monitoring device 104, perform various computations in relation to the glucose measurements 114, display information related to the glucose measurements 114 and the glucose monitoring platform 110, communicate the glucose measurements 114 to the glucose monitoring platform 110, and so forth.

Additionally, the computing device 106 may be representative of more than one device in accordance with the described techniques. In one or more scenarios, for instance, the computing device 106 may correspond to both a wearable device (e.g., a smart watch) and a mobile phone. In such scenarios, both of these devices may be capable of performing at least some of the same operations, such as to receive the glucose measurements 114 from the wearable glucose monitoring device 104, communicate them via the network 112 to the glucose monitoring platform 110, display information related to the glucose measurements 114, and so forth. Alternately or in addition, different devices may have different capabilities that other devices do not have or that are limited through computing instructions to specified devices.

In the scenario where the computing device 106 corresponds to a separate smart watch and a mobile phone, for instance, the smart watch may be configured with various sensors and functionality to measure a variety of physiological markers (e.g., heartrate, heartrate variability, breathing, rate of blood flow, and so on) and activities (e.g., steps or other exercise) of the person 102. In this scenario, the mobile phone may not be configured with these sensors and functionality, or it may include a limited amount of that functionality—although in other scenarios a mobile phone may be able to provide the same functionality. Continuing with this particular scenario, the mobile phone may have capabilities that the smart watch does not have, such as a camera to capture images associated with glucose monitoring and an amount of computing resources (e.g., battery and processing speed) that enables the mobile phone to more efficiently carry out computations in relation to the glucose measurements 114. Even in scenarios where a smart watch is capable of carrying out such computations, computing instructions may limit performance of those computations to the mobile phone so as not to burden both devices and to utilize available resources efficiently. To this extent, the computing device 106 may be configured in different ways and represent different numbers of devices than discussed herein without departing from the spirit and scope of the described techniques.

In accordance with the discussed techniques, the computing device 106 is configured to implement diabetes management feedback for improving diabetes management. In the environment 100, the computing device 106 includes glucose monitoring application 116 and storage device 118. Here, the glucose monitoring application 116 includes the diabetes management feedback generation system 120. Although illustrated as being included in computing device 106, additionally or alternatively at least some functionality of the diabetes management feedback generation system 120 is located elsewhere, such as in glucose monitoring platform 110. Further, the glucose measurements 114 and feedback library 122 are shown stored in the storage device 118. The storage device 118 may represent one or more databases and also other types of storage capable of storing the glucose measurements 114 and the feedback library 122. The feedback library 122 stores multiple feedback items (e.g., messages or message templates) that can be provided to the user 102, for example to highlight the positive impacts of recent diabetes management choices for reflection and motivation by the user 102. This feedback is also referred to as an insight or an inspirational insight.

In one or more implementations, the glucose measurements 114 and/or the feedback library 122 may be stored at least partially remote from the computing device 106, e.g., in storage of the glucose monitoring platform 110, and retrieved or otherwise accessed in connection with configuring and outputting (e.g., displaying) user interfaces for diabetes management feedback presentation. For instance, the glucose measurements 114 and/or the feedback library 122 may be generally stored in storage of the glucose monitoring platform 110 along with the glucose measurements of the user population 108 and/or the feedback library 122, and some of that data may be retrieved or otherwise accessed on an as-needed basis to display user interfaces for diabetes management feedback presentation.

Broadly speaking, the glucose monitoring application 116 is configured to support interactions with a user that enable feedback about the user's glucose to be presented. This may include, for example, obtaining the glucose measurements 114 for processing (e.g., to determine the appropriate feedback), receiving information about a user (e.g., through an onboarding process and/or user feedback), causing information to be communicated to a health care provider, causing information to be communicated to the glucose monitoring platform 110, and so forth.

In one or more implementations, the glucose monitoring application 116 also leverages resources of the glucose monitoring platform 110 in connection with diabetes management feedback for improving diabetes management. As noted above, for instance, the glucose monitoring platform 110 may be configured to store data, such as the glucose measurements 114 associated with a user (e.g., the person 102) and/or users of the user population 108, and the feedback library 122. The glucose monitoring platform 110 may also provide updates and/or additions to the glucose monitoring application 116. Further still, the glucose monitoring platform 110 may train, maintain, and/or deploy algorithms (e.g., machine learning algorithms) to generate or select feedback or to identify time periods for which feedback is provided, such as by using the wealth of data collected from the person 102 and the users of the user population 108. One or more such algorithms may require an amount of computing resources that exceeds the resources of typical, personal computing devices, e.g., mobile phones, laptops, tablet devices, and wearables, to name just a few. Nonetheless, the glucose monitoring platform 110 may include or otherwise have access to the amount of resources needed to operate such algorithms, e.g., cloud storage, server devices, virtualized resources, and so forth. The glucose monitoring platform 110 may provide a variety of resources that the glucose monitoring application 116 leverages in connection with enabling diabetes management feedback to be presented via user interfaces.

In accordance with the described techniques, the diabetes management feedback generation system 120 is configured to use the glucose measurements 114 to identify one or more feedback items in the feedback library 122 and cause output of one or more user interfaces that present the identified diabetes management feedback. The glucose monitoring application 116 may cause display of the configured user interface 124 via a display device of the computing device 106 or other display device.

As discussed above and below, a variety of diabetes management feedback (e.g., messages) may be selected or generated based on the glucose measurements 114 of the user in accordance with the described techniques. In the context of measuring glucose, e.g., continuously, and obtaining data describing such measurements, consider the following discussion of FIG. 2.

FIG. 2 depicts an example 200 of an implementation of the wearable glucose monitoring device 104 of FIG. 1 in greater detail. In particular, the illustrated example 200 includes a top view and a corresponding side view of the wearable glucose monitoring device 104. It is to be appreciated that the wearable glucose monitoring device 104 may vary in implementation from the following discussion in various ways without departing from the spirit or scope of the described techniques. As noted above, for instance, user interfaces including diabetes management feedback presentation may be configured and displayed (or otherwise output) in connection with other types of devices for glucose monitoring, such as non-wearable devices (e.g., blood glucose meters requiring finger sticks), patches, and so forth.

In this example 200, the wearable glucose monitoring device 104 is illustrated to include a sensor 202 and a sensor module 204. Here, the sensor 202 is depicted in the side view having been inserted subcutaneously into skin 206, e.g., of the person 102. The sensor module 204 is depicted in the top view as a dashed rectangle. The wearable glucose monitoring device 104 also includes a transmitter 208 in the illustrated example 200. Use of the dashed rectangle for the sensor module 204 indicates that it may be housed or otherwise implemented within a housing of the transmitter 208. In this example 200, the wearable glucose monitoring device 104 further includes adhesive pad 210 and attachment mechanism 212.

In operation, the sensor 202, the adhesive pad 210, and the attachment mechanism 212 may be assembled to form an application assembly, where the application assembly is configured to be applied to the skin 206 so that the sensor 202 is subcutaneously inserted as depicted. In such scenarios, the transmitter 208 may be attached to the assembly after application to the skin 206 via the attachment mechanism 212. Alternatively, the transmitter 208 may be incorporated as part of the application assembly, such that the sensor 202, the adhesive pad 210, the attachment mechanism 212, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, the user initiated application of the wearable glucose monitoring device 104 is nearly painless and does not require the withdrawal of blood. Moreover, the automatic sensor applicator generally enables the person 102 to embed the sensor 202 subcutaneously into the skin 206 without the assistance of a clinician or healthcare provider.

The application assembly may also be removed by peeling the adhesive pad 210 from the skin 206. It is to be appreciated that the wearable glucose monitoring device 104 and its various components as illustrated are simply one example form factor, and the wearable glucose monitoring device 104 and its components may have different form factors without departing from the spirit or scope of the described techniques.

In operation, the sensor 202 is communicatively coupled to the sensor module 204 via at least one communication channel which can be a wireless connection or a wired connection. Communications from the sensor 202 to the sensor module 204 or from the sensor module 204 to the sensor 202 can be implemented actively or passively and these communications can be continuous (e.g., analog) or discrete (e.g., digital).

The sensor 202 may be a device, a molecule, and/or a chemical which changes or causes a change in response to an event which is at least partially independent of the sensor 202. The sensor module 204 is implemented to receive indications of changes to the sensor 202 or caused by the sensor 202. For example, the sensor 202 can include glucose oxidase which reacts with glucose and oxygen to form hydrogen peroxide that is electrochemically detectable by the sensor module 204 which may include an electrode. In this example, the sensor 202 may be configured as or include a glucose sensor configured to detect analytes in blood or interstitial fluid that are indicative of diabetes management using one or more measurement techniques. In one or more implementations, the sensor 202 may also be configured to detect analytes in the blood or the interstitial fluid that are indicative of other markers, such as lactate levels, which may improve accuracy in generating various diabetes management feedback. Additionally or alternately, the wearable glucose monitoring device 104 may include additional sensors to the sensor 202 to detect those analytes indicative of the other markers.

In another example, the sensor 202 (or an additional sensor of the wearable glucose monitoring device 104—not shown) can include a first and second electrical conductor and the sensor module 204 can electrically detect changes in electric potential across the first and second electrical conductor of the sensor 202. In this example, the sensor module 204 and the sensor 202 are configured as a thermocouple such that the changes in electric potential correspond to temperature changes. In some examples, the sensor module 204 and the sensor 202 are configured to detect a single analyte, e.g., glucose. In other examples, the sensor module 204 and the sensor 202 are configured to detect multiple analytes, e.g., sodium, potassium, carbon dioxide, and glucose. Alternately or additionally, the wearable glucose monitoring device 104 includes multiple sensors to detect not only one or more analytes (e.g., sodium, potassium, carbon dioxide, glucose, and insulin) but also one or more environmental conditions (e.g., temperature). Thus, the sensor module 204 and the sensor 202 (as well as any additional sensors) may detect the presence of one or more analytes, the absence of one or more analytes, and/or changes in one or more environmental conditions.

In one or more implementations, the sensor module 204 may include a processor and memory (not shown). The sensor module 204, by leveraging the processor, may generate the glucose measurements 114 based on the communications with the sensor 202 that are indicative of the above-discussed changes. Based on these communications from the sensor 202, the sensor module 204 is further configured to generate communicable packages of data that include at least one glucose measurement 114. In one or more implementations, the sensor module 204 may configure those packages to include additional data, including, by way of example and not limitation, a sensor identifier, a sensor status, temperatures that correspond to the glucose measurements 114, measurements of other analytes that correspond to the glucose measurements 114, and so forth. It is to be appreciated that such packets may include a variety of data in addition to at least one glucose measurement 114 without departing from the spirit or scope of the described techniques.

In implementations where the wearable glucose monitoring device 104 is configured for wireless transmission, the transmitter 208 may transmit the glucose measurements 114 wirelessly as a stream of data to a computing device. Alternately or additionally, the sensor module 204 may buffer the glucose measurements 114 (e.g., in memory of the sensor module 204 and/or other physical computer-readable storage media of the wearable glucose monitoring device 104) and cause the transmitter 208 to transmit the buffered glucose measurements 114 later at various intervals, e.g., time intervals (every second, every thirty seconds, every minute, every five minutes, every hour, and so on), storage intervals (when the buffered glucose measurements 114 reach a threshold amount of data or a number of measurements), and so forth.

Having considered an example of an environment and an example of a wearable glucose monitoring device, consider now a discussion of some examples of details of the techniques for diabetes management feedback for improving diabetes management.

System Architecture

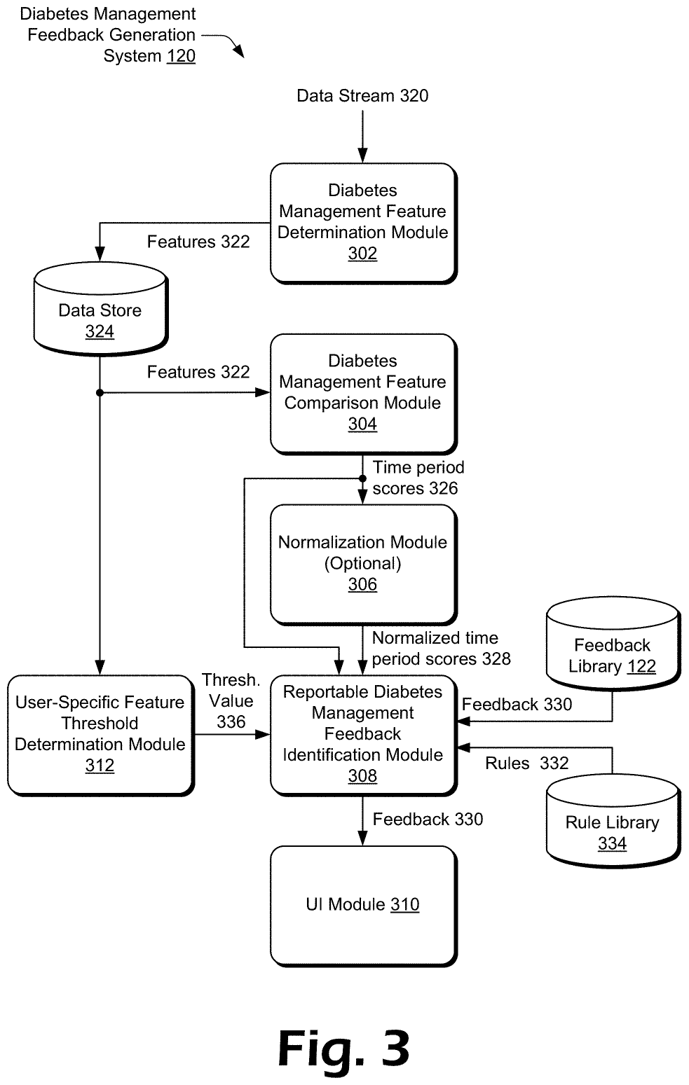

FIG. 3 is an illustration of an example architecture of a diabetes management feedback generation system 120. The diabetes management feedback generation system 120 includes a diabetes management feature determination module 302, a diabetes management feature comparison module 304, a normalization module 306 (optional), a diabetes management feedback identification module 308, a UI module 310, and a user-specific diabetes management feature threshold determination module 312. Generally, the diabetes management feedback generation system 120 analyzes the glucose measurements 114 for the user 102 and looks for patterns in the glucose measurements 114 that indicate good diabetes management by the user. Good diabetes management can be quantified in various manners, such as glucose measurements 114 staying within a particular range, glucose measurements 114 not varying by greater than a particular amount, and so forth. In one or more implementations, the diabetes management feedback generation system 120 identifies good diabetes management by identifying improvements in glucose measurements 114 for a given time period over one or more previous days, identifying a time period of the day during which glucose measurements 114 were the best (e.g., within an optimal range or closest to an optimal value), identifying sustained positive patterns (e.g., good diabetes management for a same time period in each of multiple days), or combinations thereof.

The diabetes management feature determination module 302 receives a data stream 320. In one or more implementations, the data stream 320 includes glucose measurements 114 and timestamps indicating when each of the glucose measurements 114 was taken (e.g., by wearable glucose monitoring device 104) or received (e.g., by glucose monitoring application 116). The timestamp may be provided, for example, by the wearable glucose monitoring device 104 or the glucose monitoring application 116. Additionally or alternatively, the data stream 320 includes additional data that may be used to identify good diabetes management (e.g., other data that affects glucose levels in the user 102). This additional data may also be referred to as additional data streams (e.g., the diabetes management feature determination module 302 receives multiple data streams 320 each including data from a different source or sensor).

For example, data stream 320 can include activity data, such as a number of steps walked over a particular range of time (e.g., every 10 seconds, every minute), heart rate over a particular range of time (e.g., at regular or irregular intervals, such as every 15 seconds) with timestamps, speed of movement with timestamp (e.g., at regular or irregular intervals, such as every 15 seconds), and so forth. Activity data can be received from various sources, such as wearable glucose monitoring device 104, an activity tracking application running on computing device 106, an activity or fitness tracker worn by the user 102, and so forth.

By way of another example, data stream 320 can include data regarding sleeping patterns of the user. E.g., data stream 320 can include data indicating times when the user is sleeping, the sleep state (e.g., Stage 1, Stage 2, Stage 3, or rapid eye movement (REM) sleep) of the user at particular times, and so forth.

By way of another example, data stream 320 can include data regarding user engagement with glucose monitoring application 116. E.g., this application engagement data can include timestamps of when the user 102 viewed the application as well as what screens or portions of the UI were viewed, timestamps of when the user 102 provided input to (or otherwise interacted with) the application 116 as well as what that input was, timestamps of when the user viewed or acknowledged feedback provided by diabetes management feedback generation system 120, and so forth.

By way of another example, data stream 320 can include data regarding user engagement with others of user population 108, such as via glucose monitoring platform 110. E.g., this other-user engagement data can include timestamps of when the user 102 communicated with another user as well as who that other user was, descriptions of what information was communicated with another user, and so forth.

By way of another example, data stream 320 can include meal data. E.g., this meal data can include timestamps of when the user 102 ate and what foods were consumed, timestamps of when particular types or classes of foods were consumed (e.g., vegetables, grain, meat, sweets, soda), amounts of food consumed, and so forth.

By way of another example, data stream 320 can include sleep data, such as data indicating minutes of the day when the user was sleeping. Sleep data can be received from various sources, such as wearable glucose monitoring device 104, a sleep tracking application running on computing device 106, an activity or fitness tracker worn by the user 102, and so forth.

By way of another example, data stream 320 can include medication data. E.g., this medication data can include timestamps of when user 102 took medicine and what medicine was taken (which can be used to determine whether the user 102 is taking his or her medicine at the prescribed times or intervals), indications of changes in medicines (e.g., changes in types or dosages of medicines taken), and so forth.

By way of another example, data streams 320 can include data that reflects stress management, such as heart rate variability (HRV), skin conductivity and temperature, respiration rate measurements, data from an electroencephalogram (EEG), cortisol in biofluids, volatile organic components (VOCs) emitted from the skin, and so forth.

By way of another example, data streams 320 can include data that relates to user interactions with the computing device 106, with display of the computing device 106, or with other system components that indicate level of engagement with diabetes management. Examples of such data include the number of times applications (e.g., glucose monitoring application) is opened, the time spent reviewing glucose data or previous insights or educational materials, the frequency of interactions with coaches or clinicians, and so forth.

In one or more embodiments, the diabetes management feedback generation system 120 receives data streams 320 including glucose measurements 114 and provides feedback for improving diabetes management. Furthermore, the diabetes management feedback generation system 120 optionally receives additional data in the data streams 320 that can be used to identify positive diabetes management behaviors or the impacts of these behaviors in the absence of glucose measurements 114. For example, if the user 102 only uses CGM periodically, but continues using glucose monitoring application 116 or another diabetes management application (or wears other devices that collect the additional data), the diabetes management feedback generation system 120 continues to provide feedback derived from this other data during the times when the CGM is not being used.

The diabetes management feature determination module 302 generates one or more features 322. A feature 322 refers to any value that can be computed from data in one or more data streams and that indicates whether the user has been engaging in beneficial diabetes management behaviors or lifestyle choices. A feature 322 can be a metric that is a representation or summarization of the data in the data stream 320 for a particular time period. In one or more implementations, each feature 322 is one or two values that represent or summarize the data in the data stream 320 for a particular time period, transforming the raw data obtained from the data stream 320 into a numeric indicator of the adherence to beneficial diabetes management and lifestyle choices. The diabetes management feature determination module 302 stores the generated features 322 in a data store 324 (e.g., maintained on storage device 118). The generated features 322 are maintained for a duration time that can vary by implementation. For example, the generated features 322 may be maintained for two weeks, one month, one year, and so forth.

In one or more implementations, each time period is a portion of a day (or other 24 hour interval). These time periods are chosen to capture the impacts of specific diabetes management decisions and lifestyle choices. In one or more implementations, each day is separated into multiple time periods based on when the user eats meals and when the user sleeps. For example, a day may include a first time period from midnight to 6 am (corresponding to sleep), a second time period from 6 am to noon (corresponding to after breakfast), a third time period from noon to 6 pm (corresponding to after lunch), and a fourth time period from 6 pm to midnight (corresponding to after dinner). Additionally or alternatively, additional time periods can correspond to other user actions that affect glucose levels, such as when the user exercises.

The glucose monitoring application 116 optionally provides a user interface via which the user 102 can customize the time periods to his or her typical schedule. For example, assume the user 102 typically goes to bed at 10 pm, eats breakfast at 7 am, eats lunch at noon, and eats dinner at 5 pm. These times can be provided to the glucose monitoring application 116 (e.g., by the user), which determines the time periods for the day to include a first time period from 10 pm to 7 am (corresponding to sleep), a second time period from 7 am to noon (corresponding to after breakfast), a third time period from noon to 5 pm (corresponding to after lunch), and a fourth time period from 5 pm to midnight (corresponding to after dinner). A day may be separated into other numbers of periods than four. For example, assume the user 102 typically goes to bed at 10 pm, exercises at 5 am, eats breakfast at 7 am, eats lunch at 11 am, eats an afternoon snack at 2 pm, and eats dinner at 6 pm. These times can be provided to the glucose monitoring application 116, which determines the time periods for the day to include a first time period from 10 pm to 5 am (corresponding to sleep), a second time period from 5 am to 7 am (corresponding to exercise), a third time period 7 am to 11 am (corresponding to after breakfast), a fourth time period from 11 am to 2 pm (corresponding to after lunch), a fourth time period from 2 pm to 6 pm (corresponding to snack), and a sixth time period from 6 pm to 10 pm (corresponding to after dinner).

Additionally or alternatively, different time periods for the user 102 can be automatically learned by the glucose monitoring application 116 by monitoring various data available to the glucose monitoring application 116 (e.g., exercise or sleep patterns from an activity tracker, eating patterns from a food or calorie tracking application) or detected directly (e.g., sleep onset detected by activity tracker). Various rules or criteria can be used to determine time periods based on the various data available to the glucose monitoring application 116, such as detecting sleep onset and sleep cessation from an activity tracker and using the times of sleep onset and sleep cessation to determine the time period corresponding to sleep.

In one or more implementations, the glucose monitoring application 116 uses a machine learning system to determine the different time periods for the user 102. Machine learning systems refer to a computer representation that can be tuned (e.g., trained) based on inputs to approximate unknown functions. In particular, machine learning systems can include a system that utilizes algorithms to learn from, and make predictions on, known data by analyzing the known data to learn to generate outputs that reflect patterns and attributes of the known data. For instance, a machine learning system can include decision trees, support vector machines, linear regression, logistic regression, Bayesian networks, random forest learning, dimensionality reduction algorithms, boosting algorithms, artificial neural networks, deep learning, and so forth.

The machine learning system is trained, for example, by using training data that is sets of multiple data (e.g., times of exercise, sleep, or eating during a day) and timestamps indicating when the exercise, sleep, or eating was done. Known labels are associated with the sets of multiple data indicating a time period that the data corresponds to. The machine learning system is trained by updating weights or values of layers in the machine learning system to minimize the loss between time periods generated by the machine learning system for the training data and the corresponding known labels for the training data. Various different loss functions can be used in training the machine learning system, such as cross entropy loss, mean squared error loss, and so forth.

In one or more implementations the machine learning system is trained over time as the glucose monitoring application 116 is used over time. E.g., the user can provide an indication of whether a particular time period is correct, and this indication can be used as a known label for the current time periods and used to further train the machine learning system.

Accordingly, different time periods can be established for different users. Furthermore, different time periods can be established for different days. For example, the user 102 may have different schedules on different types of days (e.g., a different schedule on weekends and holidays than on weekdays). Accordingly, the time periods for different types of days can be provided by the user 102 or determined by a machine learning system of the glucose monitoring application 116.

In one or more embodiments, the blocks of times for different time periods can vary for a user across different days. For example, a user may typically go to sleep between 11 pm and midnight, and wake up between 5:30 am and 6:30 am. For any given day, the time the user goes to sleep and the time the user wakes up can be detected using various data streams, such as data from an activity tracker worn by the user. Accordingly, the time period corresponding to sleep for the user may be 11:13 pm to 6:00 am for one day, 11:27 pm to 5:48 am the next day, 11:45 pm to 6:12 am the next day, and so forth.

In one or more embodiments, the time periods can vary for different data streams. The time periods for different data streams can be selected or identified with the intent of choosing a time window that best captures data related to the occurrence or characteristics of the behavior itself (e.g., meal choice), the impacts of the behavior on physiology (e.g., glucose, sleep quality, etc.), which may be delayed relative to the behavior, or a combination of the behavior and its impacts.

The diabetes management feature determination module 302 can generate, for each time period, any of a variety of features 322 for the data stream 320 and can generate different features 322 for different types of data in the data stream 320 (e.g., different features 322 for glucose measurements than for activity). For example, for glucose measurements the diabetes management feature determination module 302 can generate a time in range feature, such as an amount of time during the time period the glucose measurements were in an acceptable or desired range of glucose levels, e.g., between 70 milligrams per deciliter (mg/dL) and 180 mg/dL, or a narrow range between 70 mg/dL and 140 mg/dL. This acceptable or desired range can be a default range, can be a custom range set by the user or a health care professional, and so forth. By way of another example, for glucose measurements the diabetes management feature determination module 302 can generate a time below threshold feature, such as an amount of time during the time period the glucose measurements were below a particular glucose level (e.g., 250 mg/dL or 70 mg/dL). This particular glucose level can be a default level, can be a custom level set by the user or a health care professional, and so forth. By way of another example, for glucose measurements the diabetes management feature determination module 302 can generate a time above threshold feature, such as an amount of time during the time period the glucose measurements were above a particular glucose level (e.g., 250 mg/dL). This particular glucose level can be a default level, can be a custom level set by the user or a health care professional, and so forth. By way of another example, for glucose measurements the diabetes management feature determination module 302 can generate any of a variety of statistics, such as coefficient of variation for the glucose measurements in the time period (the ratio of the standard deviation to the mean for the glucose measurements in the time period), mean glucose measurement in the time period, standard deviation of the glucose measurements in the time period, and so forth. By way of another example, for glucose measurements the diabetes management feature determination module 302 can generate any of a variety of additional values, such as maximum glucose measurement in the time period, maximum glucose measurement rate of change in the time period, maximum glucose measurement rise in the time period, low blood glucose index (LBGI) in the time period, high blood glucose index (HBGI) in the time period, and so forth. By way of another example, for glucose measurements the diabetes management feature determination module 302 can generate a value indicating a rate of increase or decrease in glucose levels (e.g., a rapid rise around the time of an expected meal may allow the diabetes management feedback generation system 120 to infer that the patient consumed food with a high glycemic index, or a rapid drop from high glucose level associated with detected physical activity may allow the diabetes management feedback generation system 120 to infer that the user took action to reduce their glucose with exercise).

By way of another example, for activity data the diabetes management feature determination module 302 can generate a number of steps feature that is the number of steps taken during the time period, a heart rate reserve average or range during the time period, metabolic equivalents (METs) expended during the time period, and so forth. By way of another example, for activity data the diabetes management feature determination module 302 can generate a time in range feature, such as an amount of time during the time period the user's heart rate was in an acceptable or desired range of heart rates, e.g., between 120 beats per minute (BPM) and 170 BPM. This acceptable or desired range can be a default range, can be a custom range set by the user or a health care professional, and so forth. By way of another example, for activity data the diabetes management feature determination module 302 can generate a time above threshold feature, such as an amount of time during the time period the user's heart rate was above a particular level (e.g., 120 BPM). This particular level can be a default level, can be a custom level set by the user or a health care professional, and so forth.

By way of another example, for sleep data the diabetes management feature determination module 302 can generate a value indicating duration of sleep, number of sleep disturbances or interruptions, time spent in specific sleep states, and so forth.

The diabetes management feature comparison module 304 receives the different features 322 from the data store 324 (additionally or alternatively the features 322 may be received directly from the diabetes management feature determination module 302) and generates time period scores 326. The time period scores 326 indicate differences between the different features 322 generated for different time periods. The diabetes management feature determination module 302 generates the different features 322 for each time period in a day as discussed above. The time period scores 326 allow the diabetes management feature determination module 302 to compare the features 322 for different time periods within the same day, for the same time periods across different days or across different types of days, and so forth.

In one or more implementations, the diabetes management feature comparison module 304 compares the features 322 for each time period in a day to one another. Additionally or alternatively, the diabetes management feature comparison module 304 compares the features 322 for one or more time periods in a current day to the corresponding time periods in one or more previous days (e.g., the previous immediate week or two). The “current day” refers to a day (or other 24 hour interval) that the diabetes management feedback generation system 120 is analyzing. The current day can be, for example, the day the user 102 is currently living in, the day immediately preceding the day the user 102 is currently living in or another day in the user's past. In situations in which there are multiple types of days (e.g., weekends and holidays as one type and weekdays as another type), the diabetes management feature comparison module 304 compares the features 322 for one or more time periods in a current day (e.g., the day the user 102 is currently living in, or the immediately preceding day) to the corresponding time periods in one or more previous days of the same type (e.g., the previous immediate week or two for weekdays, the previous immediate three or four weeks for weekends and holidays).

Additionally or alternatively, the diabetes management feature comparison module 304 compares summary statistics of the features 322 computed from corresponding time periods on a set of previous days. For example, the diabetes management feature comparison module 304 can compare mean, median, interquartile range (IQR), XXth percentile, standard deviation, and so forth of features 322 computed from corresponding time periods on a set of previous days.

Additionally or alternatively, the diabetes management feature comparison module 304 compares the features 322 for one or more time periods in days of a week to the corresponding time periods in days of one or more preceding weeks. For example, for a particular diabetes management feature or feature (e.g., time in range), the feature value corresponding to a particular within-day time window (e.g., after breakfast) from a full week of data (the collection of after breakfast time windows from that week) could be compared to the same feature value for corresponding within-day time windows computed over a historical date range (e.g., the prior week or the preceding month).

In one or more implementations, the score for a time period is based on an effect size for the time period, a significance for the time period, the novelty of a generated feedback for the time period, or a combination thereof Additionally or alternatively, if there is variable duration time frame involved in the feedback, (e.g., the number of days over which a consistent pattern of positive glycemic control is detected), where the duration of the time frame indicates the relative “noteworthiness” of the feedback, this duration (or a normalized version of this duration) can serve as a score or as one component of a composite score for the time period.

The effect size for a time period refers to the difference between one time period and the other time period(s) that the one time period is being compared to. The effect size is computed in various manners, such as subtracting the features for one time period from the other or by comparing a feature from one time period to a summary statistic computed over a set of other time periods (e.g., comparing time in range after breakfast on the current day to the 90th percentile of time in range values computed for the after breakfast time period on each of the previous 14 days). For example, for a time in range feature for glucose measurements, the effect size can be computed by subtracting the amount of time in range for one time period from the amount of time in range for the other time period. E.g., if the time in range for a time period in the previous day is 120 minutes and the time in range for the corresponding time period on the current day is 150 minutes, then the effect size is 30 minutes. Additionally or alternatively, the effect size can be calculated as a percentage improvement. E.g., if the time in range for a time period in the previous day is 120 minutes and the time in range for the corresponding time period on the current day is 150 minutes, then the effect size is 25% due to 150 minutes being 25% greater than 120 minutes. Additionally or alternatively, the time in range can be identified as a percentage of the time period rather than a number of minutes. E.g., the time in range for a time period in the previous day may be 60% and the time in range for the corresponding time period on the current day may be 70%, so the effect size is 10%.

The significance of a time period refers to the difference between the one time period and the other time period(s) that the one time period is being compared to and accounts for the typical day-to-day variability for the user 102. This allows the diabetes management feedback generation system 120 to customize feedback to the typical behavior of user 102, as well as changes in the typical behavior of user 102 over time, rather than applying common thresholds or rules across all users. For example, one user may be pretty consistent with their behavior resulting in fairly consistent glucose levels, whereas another user may not be as consistent with their behavior resulting in wide swings in glucose levels. Accordingly, a smaller change in a feature would be more significant for a user that is consistent with their behavior than it would to a user that is not consistent with their behavior. The significance of the comparison accounts for these different behaviors.

The significance of a comparison can be generated in any of a variety of manners. For example, the significance of a comparison may be or include a value indicating, for a feature generated for a time period, a number of standard deviations away from the feature mean that generated feature is (e.g., the mean being calculated based on the features for a time period across multiple days, such as two weeks). Thus, the size of the standard deviation can vary on a user by user basis or over time for a given user (e.g., computed over a sliding window). Larger numbers of standard deviations away from the mean indicate a more significant difference.

The novelty of a generated feedback for the time period refers to how often or frequently a particular feedback is generated for the time period. For example, if feedback is frequently provided indicating that the time period corresponding to the breakfast time period is the best glucose levels of the day (e.g., the time period having glucose levels within an optimal range or closest to an optimal value), then the novelty score of the breakfast time period in the current day can be lower to avoid repeatedly providing the same feedback (which would be expected to be less interesting to the user). The novelty of a generated feedback for the time period is measured in any of various different manners, such as a count of how many times the feedback has been generated (e.g., over the previous two weeks or the previous month), a value indicating how frequently the feedback is generated relative to other feedbacks, and so forth.

The time period scores 326 can be output as any of a variety of different values. In one or more implementations, the time period score 326 for a time period is the effect size as determined by diabetes management feature comparison module 304. Additionally or alternatively, the time period score 326 for a time period can be a tuple including one or more of the effect size, the significance, the novelty (for each possible feedback), other indications of the differences between the different features, and so forth. Additionally or alternatively, the time period score 326 for a time period can be a value determined by combining (e.g., a weighted averaging) of one or more of the effect size, the significance, the novelty, other indications of the differences between the different features, and so forth. In such situations, a different time period score 326 can be generated for each possible feedback (e.g., as different feedbacks could have different novelties).

In one or more implementations, the time period scores 326 are provided to a normalization module 306, which adjusts the time period scores 326 resulting from different scores (e.g., the effect size, the significance, and the novelty) to a common scale or common units (e.g., a value ranging between 0 and 1). The normalization module 306 outputs the normalized data as normalized time period scores 328. This normalization can be performed using any of a variety of public or proprietary techniques. It should be noted that the normalization module 306 is optional and that normalization need not be performed in certain situations. For example, in some situations if only a single type of score (e.g., one of effect size, significance, or novelty) is used by the diabetes management feedback generation system 120 there is no need to adjust time period scores 326 to a common scale or units (although time period scores 326 may still be adjusted to a common scale or units if the effect size is computed in terms of one of several possible diabetes management features, each feature having potentially different units). By way of another example, if multiple types of scores (e.g., two or more of effect size, significance, or novelty) are used by the diabetes management feedback generation system 120 but already have a common scale or units then there is no need to adjust time period scores 326 to a common scale or units.

The diabetes management feedback identification module 308 receives time period scores, which are the time period scores 326 or the normalized time period scores 328, and selects feedback 330 to be provided to UI module 310. The diabetes management feedback identification module 308 applies any of various different rules 332 from a rule library 334 to determine which of multiple feedback items from the feedback library 122 to retrieve and provide to the UI module 310. The rule library may be stored in various locations, such as stored in the storage device 118.

Various different feedback items can be included in the feedback library 122 and provided as feedback 330, each providing feedback to the user 102. Examples of such feedback include indicating improvement of glucose levels for a time period on the current day relative to previous days, a best time period of the day (e.g., the time period of the day where glucose levels were within an optimal range or closest to an optimal value (e.g., based on any one or more of the features discussed above)), feedback identifying sustained positive patterns (e.g., a feature being satisfied for the same time period across multiple days), and so forth.

In one or more implementations, the feedback in feedback library 122 is general feedback that need not be altered based on the specific features or glucose values of the user (e.g., a message such as “Your glucose levels were best after breakfast today!”). Additionally or alternatively, the feedback in feedback library 122 includes templates to which the diabetes management feedback identification module 308 adds features or glucose values to. For example, the feedback library 122 may include a message such as “You stayed in range for the ______ night in a row!” where the underscore is filled in by the diabetes management feedback identification module 308. E.g., if the diabetes management feedback identification module 308 determines that the user was within the desired range for the three time periods corresponding to sleep, the diabetes management feedback identification module 308 replaces the blank space with “3^{rd}”.

Additional examples of feedback include: “Overnight glucose 70-140 mg/dL>40% of time for 5 days in a row,” “After-lunch peak glucose<130 mg/dL for 3 days in a row,” “After-lunch peak glucose 22 mg/dL lower than any of the past 7 days,” “18% more time 70-140 mg/dL after dinner than any of the last 7 days,” “5% more time 70-180 mg/dL after dinner than any other time period today,” “Your peak glucose after lunch was 14 mg/dL lower than any other time period today,” “Your average glucose after dinner today was 21 mg/dL lower than any of the last 7 days,” “Your time between 70 and 140 mg/dL after dinner was 4% higher than any other time period today,” and “Your time between 70 and 140 mg/dL after lunch today was 3% higher than any of the last 7 days.”

Various different rules 332 can be included in the rule library 334, and each rule 332 corresponds to one of the items of feedback in feedback library 122. Rules can be directed to different features and different time periods within a day or corresponding time periods across multiple days. For example, one rule can be which time period of the day has a highest score (e.g., the glucose measurements for a particular time period of the day were within a particular range or were below a particular glucose level for a longer duration of time than the glucose measurements during the other time periods of the day). Another rule can be whether glucose measurements for a particular time period of the day improved a threshold amount over the glucose measures for the corresponding time periods of one or more previous days (e.g., the glucose measurements were within a particular range or were below a particular glucose level for at least a threshold longer duration of time than the glucose measurements measured for the user during the time period of the one or more previous days). Another rule can be whether glucose measurements for a particular time period of the day were part of a sustained pattern of good glucose measurements (e.g., the glucose measurements for the current day as well as glucose measurements measured for the time period of one or more previous days (e.g., two to four weeks) were within a particular range or were below a particular glucose level for each of the current day and the one or more previous days).

The rules 332 can include reference to various thresholds, such as threshold values being exceeded or not being exceeded. In one or more implementations, the user-specific diabetes management feature threshold determination module 312 generates a threshold value 336, based on the features 322, that is a user-specific diabetes management feature threshold based on the distribution of the diabetes management feature observed over a historical time window (e.g., the threshold may be set to the 75th percentile of the diabetes management feature observed in a given within-day time period over the prior month) for the user 102. This threshold value 336 is then used by the diabetes management feedback identification module 308 as the threshold value in various rules 332 (for example, to identify sequences of days that meet or exceed that threshold (e.g., the sustained positive pattern)). Deriving the threshold value 336 from a user's own historical data allows the threshold value 336 to represent a level of glycemic control that represents glycemic control that is “good” relative to the typical levels observed for that user, but still achievable. The choice of summary statistic to use to set the threshold (e.g., 60th percentile versus 80th percentile) is a parameter that allows control over the balance between the “noteworthiness” of the sustained positive pattern and the achievability for that particular patient. The choice of summary statistic to use to set the threshold may be made by the user 102, by a health care professional, by an administrator or designer of the glucose monitoring application 116 or the glucose monitoring platform 110, and so forth. The threshold value 336 can also adapt to within-user changes in glycemic control over long time periods because it can be updated as the historical time window used to derive the threshold value 336 advances in time.

In one or more implementations, the diabetes management feedback identification module 308 uses a rule 332 indicating that if the score corresponding to a feature for a time period exceeds a threshold, then the rule is satisfied and feedback corresponding to that rule is selected as feedback 330. This can result in diabetes management feedback identification module 308 selecting multiple items of feedback 330 that are displayed or otherwise presented by UI module 310.

Additionally or alternatively, in situations in which for multiple rules are satisfied, diabetes management feedback identification module 308 selects one of the rules that was satisfied and selects as feedback 330 the feedback corresponding to the selected rule. The diabetes management feedback identification module 308 can select one of the multiple rules in various manners, such as randomly or pseudorandomly selecting one of the multiple rules that were satisfied. Additionally or alternatively, the diabetes management feedback identification module 308 can prioritize the multiple rules and select one of the multiple rules having a highest priority. For example, the rule having the highest priority is selected.

The diabetes management feedback identification module 308 optionally uses various criteria to determine which of the multiple rules that were satisfied to select. These criteria can be based on various factors, such as how recently a rule was satisfied, a ranking or prioritization of rules, categories of rules, how many consecutive days the rule has been satisfied, and so forth. For example, a rule that was satisfied less recently is selected over a rule that was satisfied more recently. E.g., this allows different feedback (corresponding to the different rules) to be selected as feedback 330 and avoids repeating feedback too frequently.

By way of another example, rules may correspond to different categories, such as an improvement category (e.g., rules corresponding to improvements in glucose measurements for a time period over one or more previous days), a best-of category (e.g., rules identifying a time period of the day during which glucose measurements were the best (e.g., within an optimal range or closest to an optimal value)), a sustained pattern category (e.g., rules identifying sustained positive patterns), and so forth. Rules corresponding to certain categories can be selected over rules corresponding to other categories. For example, a rule corresponding to a sustained pattern category may be selected over a rule corresponding to an improvement category, e.g., to avoid displaying feedback repeating the same improvement too frequently.

By way of another example, a rule designated (e.g., by a developer or designer of the diabetes management feedback identification module 308) to be more urgent or safety-related is selected over a rule that is less urgent or safety-related. E.g., this allows feedback corresponding to urgent or safety-related features (e.g., not staying within ranges or exceeding threshold glucose levels) to be selected over other non-urgent or non-safety-related features and display or otherwise present more critical diabetes management feedback to the user.

By way of another example, a rule designated as being higher priority (e.g., by the user 102) is selected over a rule that is designated as being lower priority (e.g., by the user 102). E.g., this allows feedback corresponding to rules that are of greater interest to the user to be displayed or otherwise presented rather than feedback corresponding to rules that are of less interest to the user.

By way of another example, a rule that has been satisfied for at least a threshold number of days in a row is selected over a rule that has not been satisfied for at least the threshold number of days in a row. E.g., this allows a good streak or good pattern (e.g., peak glucose staying below 200 mg/dL during an after lunch time period) for a number of days to be displayed or otherwise presented to the user, encouraging the user to continue with the streak or pattern.

In one or more implementations, feedback 330 is generic in nature, notifying the user 102 of particular time periods for which glucose management was good without specifying particular values indicating an amount of improvement (e.g., indicating “Your after-dinner glucose is looking great!” rather than “Your after-dinner glucose was lower than usual today”). In such situations, the diabetes management feedback identification module 308 selects feedback 330 corresponding to the time period for which multiple rules were satisfied. For example, assume that for a current day a first time period and a second time period both satisfied a time in range rule, the first time period did not satisfy a coefficient of variation rule for the glucose measurements in the time period, but the second time period did satisfy the coefficient of variation rule for the glucose measurements in the time period. In this situation, the diabetes management feedback identification module 308 selects feedback 330 corresponding to the second time period rather than the first time period because there were more rules satisfied by the second time period than the third time period. By way of another example, assume that for a current day a first time period and a second time period both satisfied a time in range rule, the first time period did not satisfy a number of steps taken rule during the time period, but the second time period did satisfy a number of steps taken rule during the time period. In this situation, the diabetes management feedback identification module 308 selects feedback 330 corresponding to the second time period rather than the first time period because there were more rules satisfied by the second time period than the third time period.

The UI module 310 receives the feedback 330 and causes the feedback 330 to be displayed or otherwise presented (e.g., at computing device 106). This display or other presentation can take various forms, such as a static text display, graphic or video display, audio presentation, combinations thereof, and so forth. In one or more implementations, different types or categories of feedback are displayed or otherwise presented in different manners. For example, feedback categories can include a category of improvements in glucose measurements for a given time period over one or more previous days, a category of which time period of the day had the best glucose measurements (e.g., which time period had glucose measurements within an optimal range or closest to an optimal value), and a category of sustained positive patterns. Feedback corresponding to these different categories can be displayed using different colors, different icons, and so forth.

In one or more implementations, the feedback 330 is ordered based on priority. For example, if multiple rules were satisfied the diabetes management feedback identification module 308 selects the highest priority feedback first as feedback 330. User input requesting additional feedback of lower priority as desired by the user 102 can be received, and the diabetes management feedback identification module 308 provides the lower priority feedback as requested by the user. E.g., this allows the highest priority feedback to be presented, then in response to a user request for additional feedback the second highest priority feedback to be presented, then in response to an additional user request for additional feedback the third highest priority feedback to be presented, and so forth.

In one or more implementations, the diabetes management feedback identification module 308 determines whether there is an improvement, based on at least one feature, in the glucose levels of the user for each of the time periods in the current day relative to one or more previous days, such as the immediately preceding week or two (these previous days are optionally days of the same type). If the diabetes management feedback identification module 308 determines that a rule indicating that the improvement for a time period satisfies a threshold (e.g., at least a certain percentage improvement), the diabetes management feedback identification module 308 selects feedback 330 corresponding to the rule.

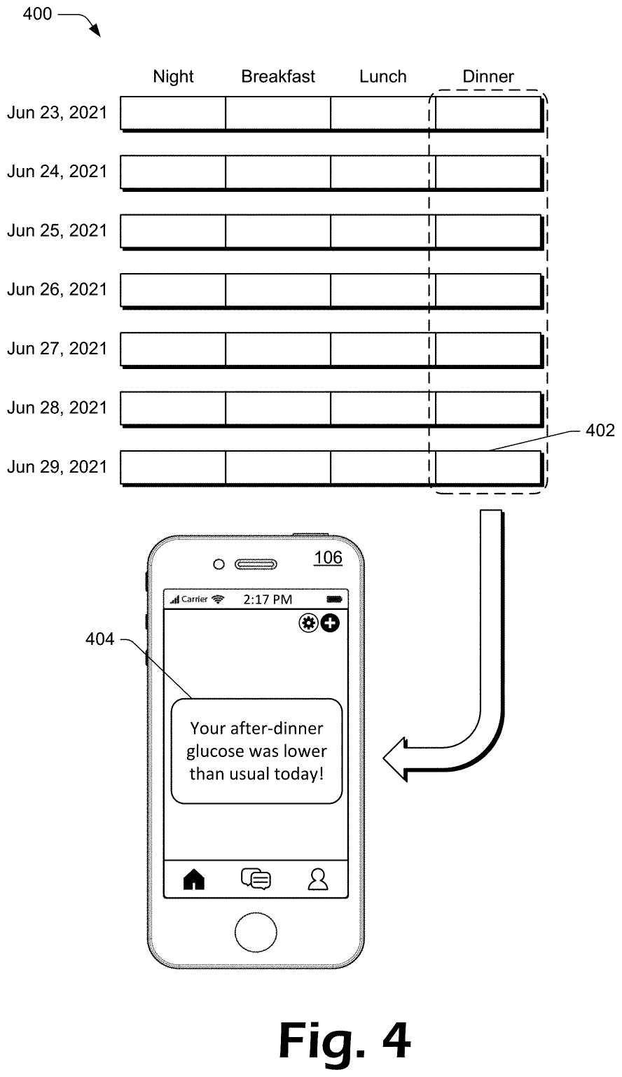

FIG. 4 illustrates an example 400 of providing feedback indicating improvement in at least one feature for a time period. The improvement in example 400 is an improvement in a time period of a current day relative to the corresponding time period in each of multiple immediately preceding days. The example 400 show multiple days (illustrated as Jun. 23, 2021-Jun. 29, 2021) each having time periods Night (e.g., while the user is sleeping), Breakfast (e.g., during and after breakfast), Lunch (e.g., during and after lunch), and Dinner (e.g., during and after dinner). A time period score is generated for time period 402 (Dinner) on the current day (June 29) that indicates the difference between one or more features for time period 402 and the same one or more features for the corresponding time period (Dinner) in the previous days June 23-June 28. In the illustrated example, the comparisons for these time periods and generated score indicate that the user's glucose level during time period 402 was lower than the typical amount for the user during the previous days June 23-June 28, which satisfies a rule 332. This determination can have been made in various manners, such as determining how many standard deviations the glucose level for time period 402 on June 29 was away from the mean generated for the corresponding time period on June 23-June 28.

The diabetes management feedback identification module 308 identifies feedback 404 and the UI module 310 causes the feedback 404 to be displayed. In the illustrated example, the feedback 404 is an inspirational insight notifying the user that his after-dinner glucose was lower than usual today. This brings the improved glucose level for a time period to the user's attention, allowing him to reflect on what changes he made to his dinner on June 29 relative to the previous days and continue those changes for better diabetes management.

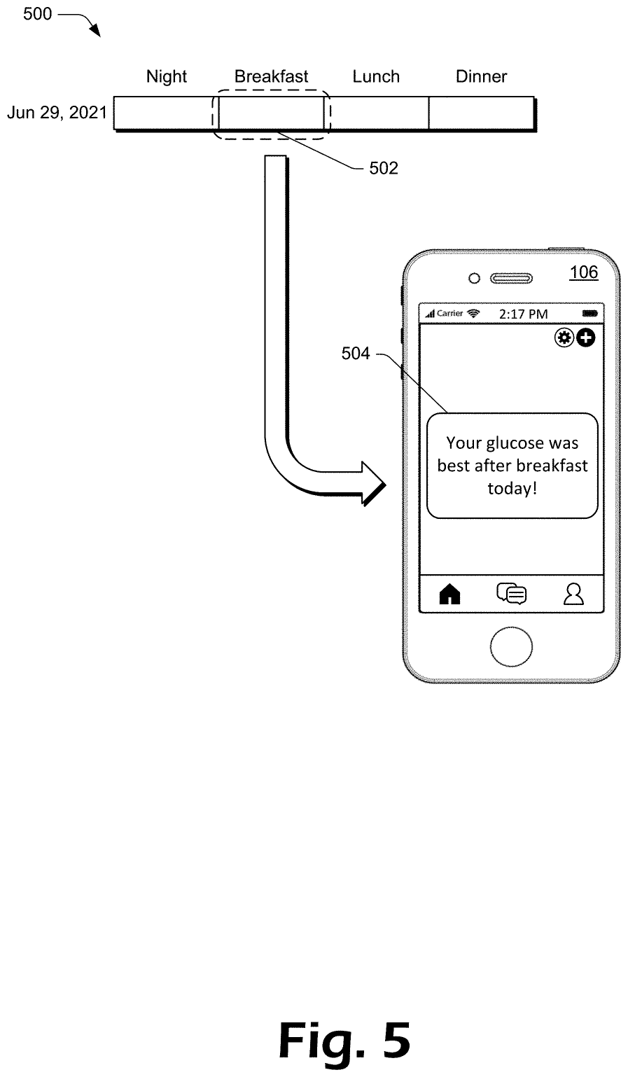

FIG. 5 illustrates an example 500 of providing feedback indicating a best time period during a day. The example 500 show a single day (illustrated as Jun. 29, 2021) having time periods Night (e.g., while the user is sleeping), Breakfast (e.g., during and after breakfast), Lunch (e.g., during and after lunch), and Dinner (e.g., during and after dinner). A time period score is generated for time period 502 (Breakfast) on the current day (June 29) that indicates the difference between one or more features for time period 502 and the same one or more features for the other three time periods on June 29. In the illustrated example, the comparisons for these time periods and generated score indicate that the user's glucose level during time period 502 was better than any other time periods on June 29, which satisfies a rule 332. This determination can have been made in various manners, such as determining that the user's glucose level was within a particular range longer than during any other time period on June 29, determining that the user's glucose level remained below a threshold level during time period 502 but not during other time periods on June 29, and so forth.

The diabetes management feedback identification module 308 identifies feedback 504 and the UI module 310 causes the feedback 504 to be displayed. In the illustrated example, the feedback 504 is an inspirational insight notifying the user that his after-breakfast glucose was better than any other time period today. This brings the improved glucose level for a time period to the user's attention, allowing him to reflect on what foods he eats during breakfast relative to the foods eaten during other time periods on June 29, and using that knowledge to change foods eaten during other time periods for better diabetes management.

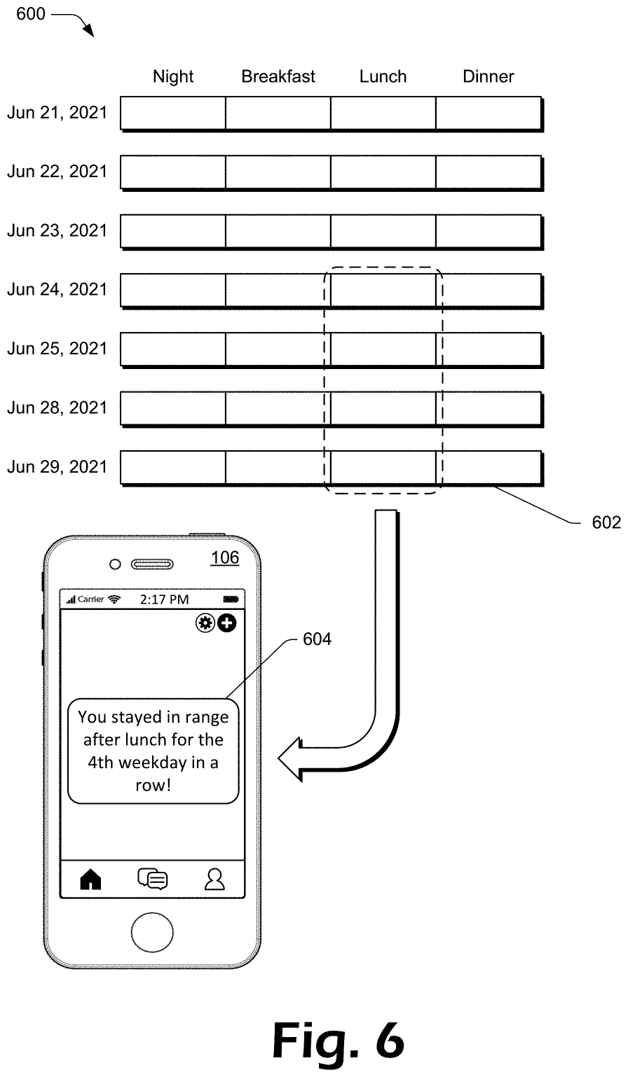

FIG. 6 illustrates an example 600 of providing feedback indicating a sustained positive pattern. The sustained positive pattern in example 600 is a pattern in a corresponding time period across multiple consecutive days of the same type (e.g., weekdays). The example 600 shows multiple days (illustrated as Jun. 21, 2021-Jun. 25, 2021, Jun. 28, 2021, and Jun. 29, 2021) each having time periods Night (e.g., while the user is sleeping), Breakfast (e.g., during and after breakfast), Lunch (e.g., during and after lunch), and Dinner (e.g., during and after dinner). A time period score is generated for time period 602 (Lunch) on the current day (June 29) that indicates the difference between one or more features for time period 602 and the same one or more features for the corresponding time period (Lunch) in each of the previous days June 21-June 25 and June 28. In the illustrated example, the comparisons for these time periods and generated score indicate that the user's glucose level during time period 602, as well as the corresponding time period on June 24, June 25, and June 28, was within a particular range, which satisfies a rule.

The diabetes management feedback identification module 308 identifies feedback 604 and the UI module 310 causes the feedback 604 to be displayed. In the illustrated example, the feedback 604 is an inspirational insight notifying the user that his overnight glucose was within the desired range after lunch for 4 weekdays in a row. This brings the sustained pattern of staying within the desired range to the user's attention, allowing him to reflect on what types of food he has been eating for lunch over the past few weekdays and continue to eat similar types of food for better diabetes management.

Returning to FIG. 3, discussions are made herein with reference to multiple time periods in a time range that is a day. Additionally or alternatively, different time periods or time ranges can be used. For example, each time period can be an entire day and the time range can be a week, a month, multiple months, a year, and so forth. By way of another example, each time period can be an entire week and the time range can be a month, multiple months, or an entire year.

Discussions are also made herein with reference to positive comparisons indicating good diabetes management and displaying or otherwise presenting feedback (e.g., inspirational insights) to motivate the user. Analogously, negative comparisons indicating poor diabetes management can be made and feedback (e.g., warnings or negative affects) displayed or otherwise presented to the user to encourage better diabetes management by the user. Negative comparisons are analogous to the techniques discussed herein, but the rules applied to the features are different. For example, feedback indicating a time period having the worst glucose levels of the day (e.g., a time period having glucose levels outside of an optimal range or furthest from an optimal value) may be displayed rather than a time period having the best glucose levels of the day (e.g., a time period having glucose levels within an optimal range or closest to an optimal value). By way of another example, feedback indicating that a time in range for a given time period was 25% less than corresponding periods in the previous days. Additional features can also be included for negative comparisons. For example, for glucose measurements the diabetes management feature determination module 302 can generate a threshold exceeded feature, such as whether a particular glucose level (e.g., 400 mg/dL) was exceeded during the time period.

In one or more implementations, the diabetes management feedback generation system 120 applies additional criteria to determining when to display or otherwise presenting negative feedback such as a warning or negative affects. This additional criterion can include identifying a pattern of the feature being satisfied on multiple occurrences, e.g., a particular time period being the worst glucose levels of the day for multiple days in a row, or the time in range for a given time period being 25% less than corresponding periods in the previous days for two days in a row. This additional criterion prevents a warning or negative affect from being displayed or otherwise presented to the user for a single bad day, avoiding unnecessarily warning the user or notifying him or her of a negative affect for a single aberration.

As discussed above, situations arise in which diabetes management feedback identification module 308 uses various criteria to determine which of multiple rules that were satisfied to select. In one or more implementations, negative comparisons are one of these criteria and operate to cause a rule corresponding to a time period that did not satisfy a negative comparison to be selected over a time period that did satisfy a negative comparison. For example, assume that for a current day a first time period and a second time period both satisfied a time in range rule, the first time period did not satisfy the negative comparison of the threshold exceeded rule (e.g., the threshold glucose level was not exceeded during the time period), but the second time period did satisfy the negative comparison of the threshold exceeded rule (e.g., the threshold glucose level was exceeded during the time period). In this situation, the diabetes management feedback identification module 308 selects a rule for the first time period rather than the second time period because there was no negative comparison satisfied for the first time period.

The diabetes management feedback generation system 120 optionally takes additional actions based on the feedback 330. In one or more implementations, these actions include notifying the glucose monitoring application 116 or the wearable glucose monitoring device 104 that the frequency with which glucose measurements 114 are produced can be reduced. For example, if the diabetes management feedback generation system 120 identifies a sustained positive pattern (e.g., glucose within a particular range) for a particular time period for at least a threshold number of days, the diabetes management feedback generation system 120 notifies the glucose monitoring application 116 or wearable glucose monitoring device 104 that the frequency with which glucose measurements 114 are produced can be reduced (e.g., from every 5 minutes to every 10 minutes), reducing the power expended to produce glucose measurements 114.

Additionally or alternatively, these actions include determining whether to recommend ongoing CGM use (e.g., starting a new sensor immediately after the current sensor expires) or whether it may be appropriate to take a break from using CGM and starting a new sensor at some later date. For example, if the diabetes management feedback generation system 120 identifies no sustained positive pattern (e.g., glucose within a particular range) for a particular time period for at least a threshold number of days, the diabetes management feedback generation system 120 recommends (e.g., via display or other presentation to the user) ongoing CGM use.

Discussions are also made herein with reference to feedback being displayed or otherwise presented to the user 102. Additionally or alternatively, the feedback is communicated to or otherwise delivered to others, such as a clinician (e.g., the user's primary care physician or nurse), a pharmacist, and so forth. This can serve to partially automate some of the manual effort of reviewing raw glucose or other diabetes management data that a clinician may have to do on their own in the absence of generated feedback. Additionally or alternatively, the time period scores 326 or normalized time period scores 328 may be provided to the clinician, pharmacist, or others, enabling them to apply their own preferred set of rules in determining which feedback should be passed along to the user.

Having discussed exemplary details of the techniques for user interfaces for glucose insight presentation, consider now some examples of procedures to illustrate additional aspects of the techniques.

Example Procedures

This section describes examples of procedures for implementing diabetes management feedback for improving diabetes management. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks.

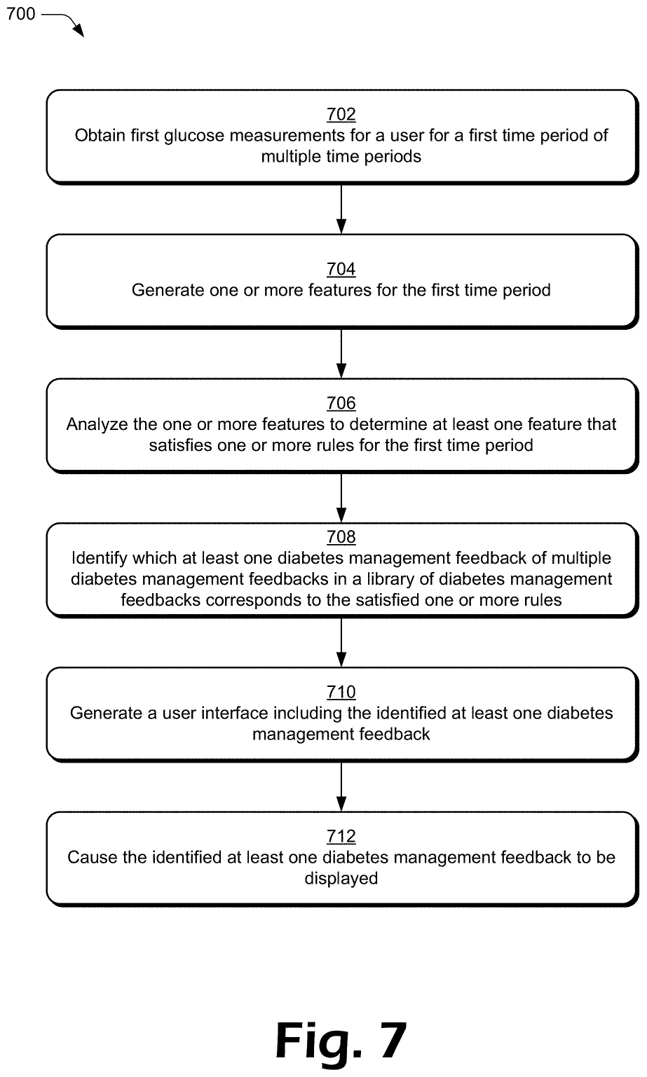

FIG. 7 depicts a procedure 700 in an example of implementing diabetes management feedback for improving diabetes management. Procedure 700 is performed, for example, by a diabetes management feedback generation system, such as the diabetes management feedback generation system 120.

First glucose measurements for a user for a first time period of multiple time periods are obtained (block 702). These first glucose measurements are obtained from a glucose sensor of, for example, a continuous glucose level monitoring system with the glucose sensor being inserted at an insertion site of the user.

One or more features for the first time period are generated (block 704). These one or more features are generated from the first glucose measurements.

The one or more features are analyzed to determine at least one feature that satisfies one or more rules for the first time period (block 706). The analysis can involve different time periods in a same 24 hour interval as the first time period, or corresponding time periods across multiple 24 hour intervals.

Which at least one diabetes management feedback of multiple diabetes management feedbacks in a library of diabetes management feedbacks corresponds to the one or more rules is identified (block 708). Different rules can correspond to different feedback, and that feedback is identified in block 708. Additionally or alternatively, different time periods can correspond to different feedback, and the feedback corresponding to the first time period is identified in block 708.

A user interface including the identified at least one diabetes management feedback and an indication of the first time period is generated (block 710). The identified at least one diabetes management feedback is caused to be displayed (block 712) or otherwise presented.

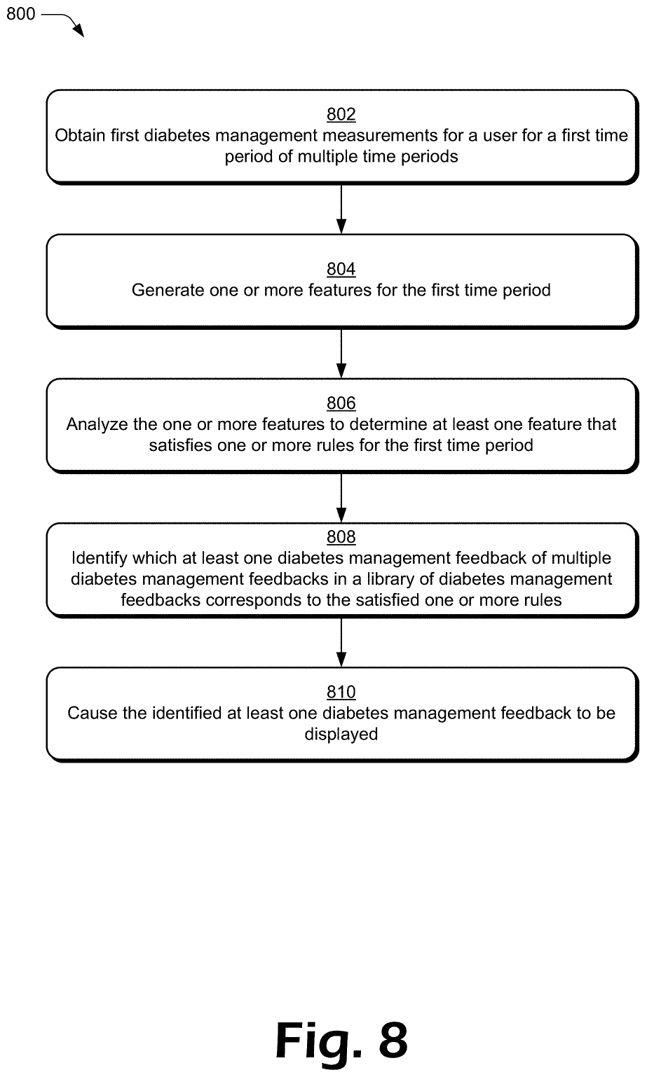

FIG. 8 depicts a procedure 800 in another example of implementing diabetes management feedback for improving diabetes management. Procedure 800 is performed, for example, by a diabetes management feedback generation system, such as the diabetes management feedback generation system 120.

First diabetes management measurements for a user for a first time period of multiple time periods are obtained (block 802). These first diabetes management measurements can be obtained from a glucose sensor of from any of a variety of other sensors as discussed above (e.g., any sensor providing data for data stream 320).

One or more features for the first time period are generated (block 804). These one or more features are generated from the first diabetes management measurements.

The one or more features are analyzed to determine at least one feature that satisfies one or more rules for the first time period (block 806). The analysis can involve different time periods in a same 24 hour interval as the first time period, or corresponding time periods across multiple 24 hour intervals.

Which at least one diabetes management feedback of multiple diabetes management feedbacks in a library of diabetes management feedbacks corresponds to the one or more rules is identified (block 808). Different rules can correspond to different feedback, and that feedback is identified in block 808. Additionally or alternatively, different time periods can correspond to different feedback, and the feedback corresponding to the first time period is identified in block 808.

The identified at least one diabetes management feedback is caused to be displayed (block 810) or otherwise presented. Additionally or alternatively, the identified diabetes management feedback can be communicated to or otherwise presented to a clinician, pharmacist, or other health care provider.

Example System and Device

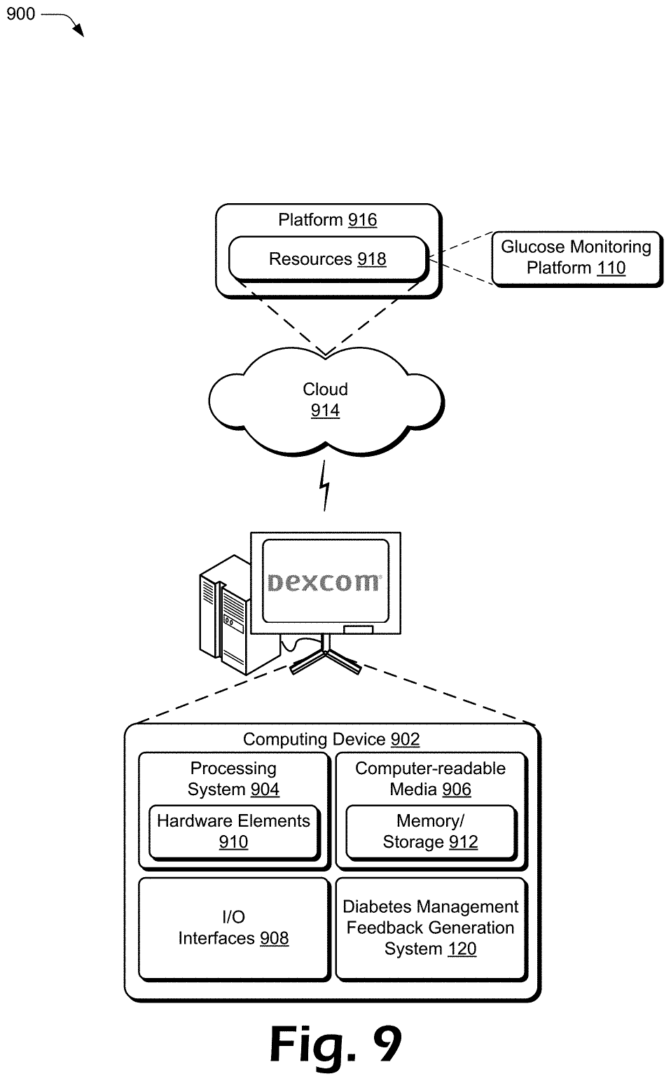

FIG. 9 illustrates an example of a system generally at 900 that includes an example of a computing device 902 that is representative of one or more computing systems and/or devices that may implement the various techniques described herein. This is illustrated through inclusion of the diabetes management feedback generation system 120. The computing device 902 may be, for example, a server of a service provider, a device associated with a client (e.g., a client device), an on-chip system, and/or any other suitable computing device or computing system.

The example computing device 902 as illustrated includes a processing system 904, one or more computer-readable media 906, and one or more input/output (I/O) interfaces 908 that are communicatively coupled, one to another. Although not shown, the computing device 902 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 904 is representative of functionality to perform one or more operations using hardware. Accordingly, the processing system 904 is illustrated as including hardware elements 910 that may be configured as processors, functional blocks, and so forth. This may include implementation in hardware as an application specific integrated circuit or other logic device formed using one or more semiconductors. The hardware elements 910 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 906 is illustrated as including memory/storage component 912. The memory/storage component 912 represents memory/storage capacity associated with one or more computer-readable media. The memory/storage component 912 may include volatile media (such as random access memory (RAM)) and/or nonvolatile media (such as read only memory (ROM), Flash memory, optical disks, magnetic disks, and so forth). The memory/storage component 912 may include fixed media (e.g., RAM, ROM, a fixed hard drive, and so on) as well as removable media (e.g., Flash memory, a removable hard drive, an optical disc, and so forth). The computer-readable media 906 may be configured in a variety of other ways as further described below.

Input/output interface(s) 908 are representative of functionality to allow a user to enter commands and information to computing device 902, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 902 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 902. By way of example, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 902, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, communication media include wired media such as a wired network or direct-wired connection, and wireless media such as acoustic, RF, infrared, and other wireless media.

As previously described, hardware elements 910 and computer-readable media 906 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 910. The computing device 902 may be configured to implement particular instructions and/or functions corresponding to the software and/or hardware modules. Accordingly, implementation of a module that is executable by the computing device 902 as software may be achieved at least partially in hardware, e.g., through use of computer-readable storage media and/or hardware elements 910 of the processing system 904. The instructions and/or functions may be executable/operable by one or more articles of manufacture (for example, one or more computing devices 902 and/or processing systems 904) to implement techniques, modules, and examples described herein.

The techniques described herein may be supported by various configurations of the computing device 902 and are not limited to the specific examples of the techniques described herein. This functionality may also be implemented all or in part through use of a distributed system, such as over a “cloud” 914 via a platform 916 as described below.

The cloud 914 includes and/or is representative of a platform 916 for resources 918. The platform 916 abstracts underlying functionality of hardware (e.g., servers) and software resources of the cloud 914. The resources 918 may include applications and/or data that can be utilized while computer processing is executed on servers that are remote from the computing device 902. Resources 918 can also include services provided over the Internet and/or through a subscriber network, such as a cellular or Wi-Fi network.

The platform 916 may abstract resources and functions to connect the computing device 902 with other computing devices. The platform 916 may also serve to abstract scaling of resources to provide a corresponding level of scale to encountered demand for the resources 918 that are implemented via the platform 916. Accordingly, in an interconnected device embodiment, implementation of functionality described herein may be distributed throughout the system 900. For example, the functionality may be implemented in part on the computing device 902 as well as via the platform 916 that abstracts the functionality of the cloud 914.

In some aspects, the techniques described herein relate to a method implemented in a diabetes management monitoring system, the method including: obtaining, from a sensor of the diabetes management monitoring system, first diabetes management measurements measured for a user for a first time period of multiple time periods of a current day; generating, from the first diabetes management measurements, one or more features for the first time period of the current day; analyzing the one or more features to determine at least one feature that satisfies one or more rules for the first time period of the current day; identifying which at least one diabetes management feedback of multiple diabetes management feedbacks in a library of diabetes management feedbacks corresponds to the satisfied one or more rules; and causing the identified at least one diabetes management feedback to be displayed.

In some aspects, the techniques described herein relate to a method, wherein each of the multiple time periods of the current day include a different multi-hour period of time during the current day.

In some aspects, the techniques described herein relate to a method, wherein the multiple time periods of the current day include an overnight time period, a breakfast time period, a lunch time period, and a dinner time period.

In some aspects, the techniques described herein relate to a method, wherein the analyzing includes: comparing the first diabetes management measurements to diabetes management measurements measured for the user during one or more additional time periods of the current day to determine whether the first diabetes management measurements were within a particular range or were below a particular level for a longer duration of time than the diabetes management measurements measured for the user during the one or more additional time periods, and determining that the first diabetes management measurements satisfy the one or more rules in response to determining that the first diabetes management measurements were within the particular range or were below a particular level for a longer duration of time than the diabetes management measurements measured for the user during the one or more additional time periods, and the identified at least one diabetes management feedback including an indication that diabetes management for the user during the current day was best during the first time period.

In some aspects, the techniques described herein relate to a method, wherein the analyzing includes: comparing the first diabetes management measurements to diabetes management measurements measured for the user during the time period of one or more previous days to determine whether the first diabetes management measurements were within a particular range or were below a particular level for at least a threshold longer duration of time than the diabetes management measurements measured for the user during the time period of the one or more previous days, and determining that the first diabetes management measurements satisfy the one or more rules in response to determining that the first diabetes management measurements were within the particular range or were below a particular level for at least the threshold longer duration of time than the diabetes management measurements measured for the user during the time period of the one or more previous days, and the identified at least one diabetes management feedback including an indication that diabetes management for the user for the time period of the current day was better than diabetes management for the user for the time period during the one or more previous days.

In some aspects, the techniques described herein relate to a method, wherein the analyzing includes: determining whether the first diabetes management measurements as well as diabetes management measurements measured for the user during the time period of one or more previous days were within a particular range or were below a particular level for each of the current day and the one or more previous days, determining that the first diabetes management measurements satisfy the one or more rules in response to determining that the first diabetes management measurements as well as diabetes management measurements measured for the user during the time period of one or more previous days were within the particular range or were below a particular level for each of the current day and the one or more previous days, and determining a total number of days by counting each of the current day and the one or more previous days, and the identified at least one diabetes management feedback including an indication that diabetes management levels for the user for the time period have been within the particular range for the total number of days.

In some aspects, the techniques described herein relate to a method, wherein the obtaining includes obtaining first activity data from an activity tracker worn by the user, and the generating including generating the one or more features for the first time period from both the first diabetes management measurements and the first activity data.

In some aspects, the techniques described herein relate to a method, wherein the identifying includes identifying two or more to rules satisfied by the first diabetes management measurements, the method further including determining priorities for each of the two or more rules, and identifying as the at least one diabetes management feedback, the diabetes management feedback corresponding to one of the two or more rules having a highest priority.

In some aspects, the techniques described herein relate to a device including a diabetes management monitoring system, the device including: a library of diabetes management feedbacks; a display device; a diabetes management feature determination module, implemented at least in part in hardware, to obtain, from a sensor, first diabetes management measurements measured for a user for a first time period of multiple time periods of a current day; a diabetes management feature comparison module, implemented at least in part in hardware, to generate, from the first diabetes management measurements, one or more features for the first time period of the current day and analyze the one or more features to determine at least one feature that satisfies one or more rules for the first time period of the current day; and a reportable diabetes management feedback identification module, implemented at least in part in hardware, to identify which at least one diabetes management feedback of multiple diabetes management feedbacks in the library of diabetes management feedbacks corresponds to the satisfied one or more rules, generate a user interface including the identified at least one diabetes management feedback and an indication of the first time period, and cause the identified at least one diabetes management feedback to be displayed.

In some aspects, the techniques described herein relate to a device, wherein each of the multiple time periods of the current day includes a different multi-hour period of time during a 24 hour interval.

In some aspects, the techniques described herein relate to a device, wherein the multiple time periods of the current day include an overnight time period, a breakfast time period, a lunch time period, and a dinner time period.

In some aspects, the techniques described herein relate to a device, wherein the diabetes management feature comparison module is to analyze the one or more features by: comparing the first diabetes management measurements to diabetes management measurements measured for the user during one or more additional time periods of the current day to determine whether the first diabetes management measurements were within a particular range or were below a particular level for a longer duration of time than the diabetes management measurements measured for the user during the one or more additional time periods, and determining that the first diabetes management measurements satisfy the one or more rules in response to determining that the first diabetes management measurements were within the particular range or were below a particular level for a longer duration of time than the diabetes management measurements measured for the user during the one or more additional time periods, and the identified at least one diabetes management feedback including an indication that diabetes management for the user during the current day was best during the first time period.

In some aspects, the techniques described herein relate to a device, wherein the diabetes management feature comparison module is to analyze the one or more features by: comparing the first diabetes management measurements to diabetes management measurements measured for the user during the time period of one or more previous days to determine whether the first diabetes management measurements were within a particular range or were below a particular glucose level for at least a threshold longer duration of time than the diabetes management measurements measured for the user during the time period of the one or more previous days, and determining that the first diabetes management measurements satisfy the one or more rules in response to determining that the first diabetes management measurements were within the particular range or were below a particular level for at least the threshold longer duration of time than the diabetes management measurements measured for the user during the time period of the one or more previous days, and the identified at least one diabetes management feedback including an indication that diabetes management for the user for the time period of the current day was better than diabetes management for the user for the time period during the one or more previous days.

In some aspects, the techniques described herein relate to a device, wherein the diabetes management feature comparison module is to analyze the one or more features by: determining whether the first diabetes management measurements as well as diabetes management measurements measured for the user during the time period of one or more previous days were within a particular range or were below a particular level for each of the current day and the one or more previous days, determining that the first diabetes management measurements satisfy the one or more rules in response to determining that the first diabetes management measurements as well as diabetes management measurements measured for the user during the time period of one or more previous days were within the particular range or were below a particular level for each of the current day and the one or more previous days, and determining a total number of days by counting each of the current day and the one or more previous days, and the identified at least one diabetes management feedback including an indication that diabetes management measurement levels for the user for the time period have been within the particular range for the total number of days.

In some aspects, the techniques described herein relate to a device, wherein the diabetes management feature determination module is further to obtain first activity data from an activity tracker worn by the user, and the diabetes management feature comparison module is to generate the one or more features for the first time period from both the first diabetes management measurements and the first activity data.

In some aspects, the techniques described herein relate to a device, wherein the reportable diabetes management feedback identification module is to identify two or more to rules satisfied by the first diabetes management measurements, determine priorities for each of the two or more rules, and identify as the identified at least one diabetes management feedback the diabetes management feedback corresponding to one of the two or more rules having a highest priority.

In some aspects, the techniques described herein relate to a computing device including: a processor; a library storing multiple diabetes management feedbacks; a display device; and computer-readable storage media having stored thereon multiple instructions of an application that, responsive to execution by the processor, cause the processor to: obtain, from a sensor, first diabetes management measurements measured for a user for a first time period of multiple time periods of a current day; generate, from the first diabetes management measurements, one or more features for the first time period of the current day; analyze the one or more features to determine whether at least one feature satisfies one or more rules for the first time period of the current day; identify which at least one diabetes management feedback of multiple diabetes management feedbacks in a library of diabetes management feedbacks corresponds to the time period of the current day; and cause the identified at least one diabetes management feedback to be displayed.

In some aspects, the techniques described herein relate to a computing device, wherein each of the multiple time periods of the current day includes a different multi-hour period of time during a 24 hour interval.

In some aspects, the techniques described herein relate to a computing device, wherein the multiple time periods of the current day include an overnight time period, a breakfast time period, a lunch time period, an exercise time period, and a dinner time period.

In some aspects, the techniques described herein relate to a computing device, wherein: to obtain the first diabetes management measurements is further to obtain first activity data from an activity tracker worn by the user, and to generate the one or more features is to generate the one or more features for the first time period from both the first diabetes management measurements and the first activity data.

In some aspects, the techniques described herein relate to a method implemented in a continuous glucose level monitoring system, the method including: obtaining, from a glucose sensor of the continuous glucose level monitoring system, first glucose measurements measured for a user for a first time period of multiple time periods of a current day, the glucose sensor being inserted at an insertion site of the user; generating, from the first glucose measurements, one or more features for the first time period of the current day; analyzing the one or more features to determine at least one feature that satisfies one or more rules for the first time period of the current day; identifying which at least one diabetes management feedback of multiple diabetes management feedbacks in a library of diabetes management feedbacks corresponds to the satisfied one or more rules; generating a user interface including the identified at least one diabetes management feedback and an indication of the first time period; and causing the identified at least one diabetes management feedback to be displayed.

In some aspects, the techniques described herein relate to a method, wherein each of the multiple time periods of the current day includes a different multi-hour period of time during the current day.

In some aspects, the techniques described herein relate to a method, wherein the multiple time periods of the current day include an overnight time period, a breakfast time period, a lunch time period, and a dinner time period.

In some aspects, the techniques described herein relate to a method, wherein the analyzing includes: comparing the first glucose measurements to glucose measurements measured for the user during one or more additional time periods of the current day to determine whether the first glucose measurements were within a particular range or were below a particular glucose level for a longer duration of time than the glucose measurements measured for the user during the one or more additional time periods, and determining that the first glucose measurements satisfy the one or more rules in response to determining that the first glucose measurements were within the particular range or were below a particular glucose level for a longer duration of time than the glucose measurements measured for the user during the one or more additional time periods, and the identified at least one diabetes management feedback includes an indication that glucose levels for the user during the current day were best during the first time period.

In some aspects, the techniques described herein relate to a method, wherein the analyzing includes: comparing the first glucose measurements to glucose measurements measured for the user during the time period of one or more previous days to determine whether the first glucose measurements were within a particular range or were below a particular glucose level for at least a threshold longer duration of time than the glucose measurements measured for the user during the time period of the one or more previous days, and determining that the first glucose measurements satisfy the one or more rules in response to determining that the first glucose measurements were within the particular range or were below a particular glucose level for at least the threshold longer duration of time than the glucose measurements measured for the user during the time period of the one or more previous days, and the identified at least one diabetes management feedback includes an indication that glucose levels for the user for the time period of the current day were better than glucose levels for the user for the time period during the one or more previous days.

In some aspects, the techniques described herein relate to a method, wherein the analyzing includes determining whether the first glucose measurements as well as glucose measurements measured for the user during the time period of one or more previous days were within a particular range or were below a particular glucose level for each of the current day and the one or more previous days, determining that the first glucose measurements satisfy the one or more rules in response to determining that the first glucose measurements as well as glucose measurements measured for the user during the time period of one or more previous days were within the particular range or were below a particular glucose level for each of the current day and the one or more previous days, and determining a total number of days by counting each of the current day and the one or more previous days, and the identified at least one diabetes management feedback includes an indication that glucose levels for the user for the time period have been within the particular range for the total number of days.

In some aspects, the techniques described herein relate to a method, wherein the obtaining includes obtaining first activity data from an activity tracker worn by the user, and the generating includes generating the one or more features for the first time period from both the first glucose measurements and the first activity data.

In some aspects, the techniques described herein relate to a method, wherein the identifying includes: identifying two or more to rules satisfied by the first glucose measurements, the method further including determining priorities for each of the two or more rules, and identifying as the at least one diabetes management feedback, the diabetes management feedback corresponding to one of the two or more rules having a highest priority.

In some aspects, the techniques described herein relate to a device including a continuous glucose level monitoring system, the device including: a library of diabetes management feedbacks; a display device; a diabetes management feature determination module, implemented at least in part in hardware, to obtain, from a glucose sensor, first glucose measurements measured for a user for a first time period of multiple time periods of a current day, the glucose sensor being inserted at an insertion site of the user; a diabetes management feature comparison module, implemented at least in part in hardware, to generate, from the first glucose measurements, one or more features for the first time period of the current day and analyze the one or more features to determine at least one feature that satisfies one or more rules for the first time period of the current day; and a reportable diabetes management feedback identification module, implemented at least in part in hardware, to identify which at least one diabetes management feedback of multiple diabetes management feedbacks in the library of diabetes management feedbacks corresponds to the satisfied one or more rules, generate a user interface including the identified at least one diabetes management feedback and an indication of the first time period, and cause the identified at least one diabetes management feedback to be displayed.

In some aspects, the techniques described herein relate to a device, wherein each of the multiple time periods of the current day includes a different multi-hour period of time during a 24 hour interval.

In some aspects, the techniques described herein relate to a device, wherein the multiple time periods of the current day include an overnight time period, a breakfast time period, a lunch time period, and a dinner time period.

In some aspects, the techniques described herein relate to a device, wherein the diabetes management feature comparison module is to analyze the one or more features by: comparing the first glucose measurements to glucose measurements measured for the user during one or more additional time periods of the current day to determine whether the first glucose measurements were within a particular range or were below a particular glucose level for a longer duration of time than the glucose measurements measured for the user during the one or more additional time periods, and determining that the first glucose measurements satisfy the one or more rules in response to determining that the first glucose measurements were within the particular range or were below a particular glucose level for a longer duration of time than the glucose measurements measured for the user during the one or more additional time periods, and the identified at least one diabetes management feedback includes an indication that glucose levels for the user during the current day were best during the first time period.

In some aspects, the techniques described herein relate to a device, wherein the diabetes management feature comparison module is to analyze the one or more features by: comparing the first glucose measurements to glucose measurements measured for the user during the time period of one or more previous days to determine whether the first glucose measurements were within a particular range or were below a particular glucose level for at least a threshold longer duration of time than the glucose measurements measured for the user during the time period of the one or more previous days, and determining that the first glucose measurements satisfy the one or more rules in response to determining that the first glucose measurements were within the particular range or were below a particular glucose level for at least the threshold longer duration of time than the glucose measurements measured for the user during the time period of the one or more previous days, and the identified at least one diabetes management feedback includes an indication that glucose levels for the user for the time period of the current day were better than glucose levels for the user for the time period during the one or more previous days.

In some aspects, the techniques described herein relate to a device, wherein the diabetes management feature comparison module is to analyze the one or more features by: determining whether the first glucose measurements as well as glucose measurements measured for the user during the time period of one or more previous days were within a particular range or were below a particular glucose level for each of the current day and the one or more previous days, determining that the first glucose measurements satisfy the one or more rules in response to determining that the first glucose measurements as well as glucose measurements measured for the user during the time period of one or more previous days were within the particular range or were below a particular glucose level for each of the current day and the one or more previous days, and determining a total number of days by counting each of the current day and the one or more previous days, and the identified at least one diabetes management feedback includes an indication that glucose levels for the user for the time period have been within the particular range for the total number of days.

In some aspects, the techniques described herein relate to a device, wherein: the diabetes management feature determination module is further to obtain first activity data from an activity tracker worn by the user, and the diabetes management feature comparison module is to generate the one or more features for the first time period from both the first glucose measurements and the first activity data.

In some aspects, the techniques described herein relate to a device, wherein the reportable diabetes management feedback identification module is to: identify two or more to rules satisfied by the first glucose measurements, determine priorities for each of the two or more rules, and identify as the at least one diabetes management feedback the diabetes management feedback corresponding to one of the two or more rules having a highest priority.

In some aspects, the techniques described herein relate to a computing device including: a processor; a library storing multiple diabetes management feedbacks; a display device; and computer-readable storage media having stored thereon multiple instructions of an application that, responsive to execution by the processor, cause the processor to: obtain, from a glucose sensor of a continuous glucose level monitoring system, first glucose measurements measured for a user for a first time period of multiple time periods of a current day, the glucose sensor being inserted at an insertion site of the user; generate, from the first glucose measurements, one or more features for the first time period of the current day; analyze the one or more features to determine whether at least one feature satisfies one or more rules for the first time period of the current day; identify which at least one diabetes management feedback of multiple diabetes management feedbacks in a library of diabetes management feedbacks corresponds to the time period of the current day; generate a user interface including the identified at least one diabetes management feedback, the identified diabetes management feedback including an indication of the first time period; and cause the identified at least one diabetes management feedback to be displayed on the display device.

In some aspects, the techniques described herein relate to a computing device, wherein each of the multiple time periods of the current day includes a different multi-hour period of time during a 24 hour interval.

In some aspects, the techniques described herein relate to a computing device, wherein the multiple time periods of the current day include an overnight time period, a breakfast time period, a lunch time period, an exercise time period, and a dinner time period.

In some aspects, the techniques described herein relate to a computing device, wherein: to obtain the first glucose measurements is further to obtain first activity data from an activity tracker worn by the user, and to generate the one or more features is to generate the one or more features for the first time period from both the first glucose measurements and the first activity data.

Conclusion

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A method implemented in a continuous glucose level monitoring system, the method comprising:
obtaining, from a glucose sensor of the continuous glucose level monitoring system, first glucose measurements measured for a user for a first time period of multiple time periods of a current day, the glucose sensor being inserted at an insertion site of the user;
generating, from the first glucose measurements, one or more features for the first time period of the current day;
analyzing the one or more features to determine at least one feature that satisfies one or more rules for the first time period of the current day;
identifying which at least one diabetes management feedback of multiple diabetes management feedbacks in a library of diabetes management feedbacks corresponds to the satisfied one or more rules;
generating a user interface including the identified at least one diabetes management feedback and an indication of the first time period; and
causing the identified at least one diabetes management feedback to be displayed.

2. The method of claim 1, wherein each of the multiple time periods of the current day comprises a different multi-hour period of time during the current day.

3. The method of claim 1, wherein the multiple time periods of the current day include an overnight time period, a breakfast time period, a lunch time period, and a dinner time period.

4. The method of claim 1, wherein the analyzing includes:
comparing the first glucose measurements to glucose measurements measured for the user during one or more additional time periods of the current day to determine whether the first glucose measurements were within a particular range or were below a particular glucose level for a longer duration of time than the glucose measurements measured for the user during the one or more additional time periods, and
determining that the first glucose measurements satisfy the one or more rules in response to determining that the first glucose measurements were within the particular range or were below a particular glucose level for a longer duration of time than the glucose measurements measured for the user during the one or more additional time periods, and the identified at least one diabetes management feedback includes an indication that glucose levels for the user during the current day were best during the first time period.

5. The method of claim 1, wherein the analyzing includes:
comparing the first glucose measurements to glucose measurements measured for the user during the time period of one or more previous days to determine whether the first glucose measurements were within a particular range or were below a particular glucose level for at least a threshold longer duration of time than the glucose measurements measured for the user during the time period of the one or more previous days, and
determining that the first glucose measurements satisfy the one or more rules in response to determining that the first glucose measurements were within the particular range or were below a particular glucose level for at least the threshold longer duration of time than the glucose measurements measured for the user during the time period of the one or more previous days, and the identified at least one diabetes management feedback includes an indication that glucose levels for the user for the time period of the current day were better than glucose levels for the user for the time period during the one or more previous days.

6. The method of claim 1, wherein the analyzing includes:
determining whether the first glucose measurements as well as glucose measurements measured for the user during the time period of one or more previous days were within a particular range or were below a particular glucose level for each of the current day and the one or more previous days,
determining that the first glucose measurements satisfy the one or more rules in response to determining that the first glucose measurements as well as glucose measurements measured for the user during the time period of one or more previous days were within the particular range or were below a particular glucose level for each of the current day and the one or more previous days, and
determining a total number of days by counting each of the current day and the one or more previous days, and the identified at least one diabetes management feedback includes an indication that glucose levels for the user for the time period have been within the particular range for the total number of days.

7. The method of claim 1, wherein the obtaining includes obtaining first activity data from an activity tracker worn by the user, and the generating includes generating the one or more features for the first time period from both the first glucose measurements and the first activity data.

8. The method of claim 1, wherein the identifying includes:
identifying two or more to rules satisfied by the first glucose measurements, the method further including determining priorities for each of the two or more rules, and
identifying, as the at least one diabetes management feedback, the diabetes management feedback corresponding to one of the two or more rules having a highest priority.

9. A device including a continuous glucose level monitoring system, the device comprising:
a library of diabetes management feedbacks;
a display device;
a diabetes management feature determination module, implemented at least in part in hardware, to obtain, from a glucose sensor, first glucose measurements measured for a user for a first time period of multiple time periods of a current day, the glucose sensor being inserted at an insertion site of the user;
a diabetes management feature comparison module, implemented at least in part in hardware, to generate, from the first glucose measurements, one or more features for the first time period of the current day and analyze the one or more features to determine at least one feature that satisfies one or more rules for the first time period of the current day; and
a reportable diabetes management feedback identification module, implemented at least in part in hardware, to identify which at least one diabetes management feedback of multiple diabetes management feedbacks in the library of diabetes management feedbacks corresponds to the satisfied one or more rules, generate a user interface including the identified at least one diabetes management feedback and an indication of the first time period, and cause the identified at least one diabetes management feedback to be displayed.

10. The device of claim 9, wherein each of the multiple time periods of the current day comprises a different multi-hour period of time during a 24 hour interval.

11. The device of claim 9, wherein the multiple time periods of the current day include an overnight time period, a breakfast time period, a lunch time period, and a dinner time period.

12. The device of claim 9, wherein the diabetes management feature comparison module is to analyze the one or more features by:
comparing the first glucose measurements to glucose measurements measured for the user during one or more additional time periods of the current day to determine whether the first glucose measurements were within a particular range or were below a particular glucose level for a longer duration of time than the glucose measurements measured for the user during the one or more additional time periods, and
determining that the first glucose measurements satisfy the one or more rules in response to determining that the first glucose measurements were within the particular range or were below a particular glucose level for a longer duration of time than the glucose measurements measured for the user during the one or more additional time periods, and the identified at least one diabetes management feedback includes an indication that glucose levels for the user during the current day were best during the first time period.

13. The device of claim 9, wherein the diabetes management feature comparison module is to analyze the one or more features by:
comparing the first glucose measurements to glucose measurements measured for the user during the time period of one or more previous days to determine whether the first glucose measurements were within a particular range or were below a particular glucose level for at least a threshold longer duration of time than the glucose measurements measured for the user during the time period of the one or more previous days, and
determining that the first glucose measurements satisfy the one or more rules in response to determining that the first glucose measurements were within the particular range or were below a particular glucose level for at least the threshold longer duration of time than the glucose measurements measured for the user during the time period of the one or more previous days, and the identified at least one diabetes management feedback includes an indication that glucose levels for the user for the time period of the current day were better than glucose levels for the user for the time period during the one or more previous days.

14. The device of claim 9, wherein the diabetes management feature comparison module is to analyze the one or more features by:
determining whether the first glucose measurements as well as glucose measurements measured for the user during the time period of one or more previous days were within a particular range or were below a particular glucose level for each of the current day and the one or more previous days, determining that the first glucose measurements satisfy the one or more rules in response to determining that the first glucose measurements as well as glucose measurements measured for the user during the time period of one or more previous days were within the particular range or were below a particular glucose level for each of the current day and the one or more previous days, and
determining a total number of days by counting each of the current day and the one or more previous days, and the identified at least one diabetes management feedback includes an indication that glucose levels for the user for the time period have been within the particular range for the total number of days.

15. The device of claim 9, wherein:
the diabetes management feature determination module is further to obtain first activity data from an activity tracker worn by the user, and
the diabetes management feature comparison module is to generate the one or more features for the first time period from both the first glucose measurements and the first activity data.

16. The device of claim 9, wherein the reportable diabetes management feedback identification module is to:
identify two or more to rules satisfied by the first glucose measurements,
determine priorities for each of the two or more rules, and
identify as the at least one diabetes management feedback the diabetes management feedback corresponding to one of the two or more rules having a highest priority.

17. A computing device comprising:
a processor;
a library storing multiple diabetes management feedbacks;
a display device; and
computer-readable storage media having stored thereon multiple instructions of an application that, responsive to execution by the processor, cause the processor to:
obtain, from a sensor, first diabetes management measurements measured for a user for a first time period of multiple time periods of a current day;
generate, from the first diabetes management measurements, one or more features for the first time period of the current day;
analyze the one or more features to determine whether at least one feature satisfies one or more rules for the first time period of the current day;
identify which at least one diabetes management feedback of multiple diabetes management feedbacks in a library of diabetes management feedbacks corresponds to the time period of the current day; and
cause the identified at least one diabetes management feedback to be displayed.

18. The computing device of claim 17, wherein each of the multiple time periods of the current day comprises a different multi-hour period of time during a 24 hour interval.

19. The computing device of claim 17, wherein the multiple time periods of the current day include an overnight time period, a breakfast time period, a lunch time period, an exercise time period, and a dinner time period.

20. The computing device of claim 17, wherein:
to obtain the first diabetes management measurements is further to obtain first activity data from an activity tracker worn by the user, and
to generate the one or more features is to generate the one or more features for the first time period from both the first diabetes management measurements and the first activity data.
